(19) United States
(12) Patent Application Publication
(10) Pub. No.: US 2022/0356162 A1
(43) Pub. Date: Nov. 10, 2022

(54) Heterocyclic Compounds as Lipoxygenase Inhibitors

(71) Applicants: Dr. Reddy's Institute of Life Sciences, Hyderabad (IN); University of Hyderabad, Hyderabad (IN)

(72) Inventors: Reddanna Pallu, Hyderabad (IN); Kumar Reddy Kakularam, Piler (IN); Nooruddin Khan, Hyderabad (IN); Harshavardhan Dattarao Bhuktar, Lakh, Aundha (Nagnath) (IN); Manojit Pal, Hyderabad (IN); Sharda Shukla, Satna (IN); Naireen Fatima, Hyderabad (IN)

(73) Assignees: Dr. Reddy's Institute of Life Sciences, Hyderabad (IN); University of Hyderabad, Hyderabad (IN)

(21) Appl. No.: 17/620,657

(22) PCT Filed: May 20, 2020
(86) PCT No.: PCT/IN2020/050455

§ 371 Date: Dec. 17, 2021

(30) Foreign Application Priority Data
Jun. 19, 2019 (IN) 201941024295

(51) Int. Cl. C07D 311/30

(52) U.S. Cl. CPC C07D 311/30

(57) ABSTRACT

The present invention relates to a compound of formula (I):

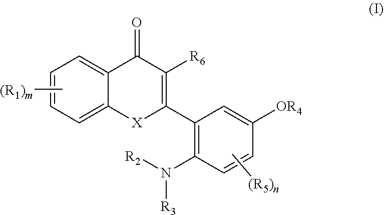

or a pharmaceutically acceptable salt or a stereoisomer thereof, wherein X, R₁, R₂, R₃, R₄, R₅, R₆, m and n are as defined in the detailed description. The present invention also relates to pharmaceutical compositions containing one or more compounds of the formula (I), as well as methods of treating or preventing diseases and/or disorders mediated by aberrant or undesired expression of one or more lipoxygenases (LOXs) using such compounds or compositions.

### RELATED APPLICATION

This application claims the benefit of Indian provisional application number 201941024295, filed on Jun. 19, 2019; the contents of which are hereby incorporated by reference in their entirety.

### FIELD OF THE INVENTION

The present invention relates to lipoxygenase (LOX) inhibitors and particularly to heterocyclic compounds, compositions and their use for the treatment of lipoxygenase mediated diseases and/or disorders.

### BACKGROUND OF THE INVENTION

Arachidonic acid, the most dominant polyunsaturated fatty acid in mammalian systems, is mainly oxygenated by Cyclooxygenase (COX) and Lipoxygenase (LOX) pathways. While COX pathway leads to the formation of prostaglandins (PGs) and thromboxanes, LOX pathway majorly produces leukotrienes (LTs). These compounds, collectively termed as eicosanoids, are potent biologically active molecules with bewildering variety of actions on different processes. The uncontrolled production of these eicosanoids leads to the manifestation of various inflammatory diseases like arthritis, psoriasis, coronary heart diseases, allergy, asthma, certain forms of cancer and even Alzheimer's disease. As a result, COX and LOX have become the natural targets for the development of drugs for a variety of inflammatory disorders.

Lipoxygenases (LOX) represent a family of non-heme iron containing dioxygenases that catalyze the regioselective and stereo selective deoxygenation of fatty acid substrates containing one or more (Z,Z)-1,4-pentadiene moieties. Based on the phylogenetic tree, the mammalian lipoxygenases are classified in to four groups: i) 5-LOX ii) platelet-type 12-LOX, iii) 15/12-LOX (reticulocyte-type 15 LOX-1 and leukocyte type 12-LOX) and iv) epidermis-type LOX (12R-LOX, 15-LOX-2, 8-LOX, epidermis-type LOX-3). The primary reaction products are hydroperoxides of conjugated (E,Z)-dienes that are further metabolized to various bioactive lipid mediators such as leukotrienes, lipoxins, hydroxyeicosatetraenoic acids (HETEs) and hepoxilins. Among these the leukotrienes are the major mediators of allergy and asthma and hence the 5-LOX inhibitors like Zileuton and the leukotriene receptor antagonists like montelukast are being used for the treatment of allergy and asthma. 12R-LOX, the targeted enzyme in psoriasis and other skin disorders: A recognized feature of psoriasis and other proliferative dermatoses is the accumulation in the skin of the unusual arachidonic acid metabolite, 12R-hydroxyeicosatetraenoic acid (12R-HETE), a product of 12R-Lipoxygenase (12R-LOX). In mammals, a 12R-LOX was discovered simultaneously in both human and mouse skin. Both enzymes are about 80 kDa in size with 86% similarity and both have a proline-rich 31 amino acid inserts. The role of 12R-LOX in normal skin development is implied from its spatial and temporal expression patterns in the mouse embryo. Microarray studies, in humans, reveal that 12R-LOX was pathologically over-expressed during the Psoriasis and other proliferative skin dermatoses. In addition, genetic studies in six families affected by NCIE (Non-bullous congenital ichthyosiform erythroderma) categorically indicate that 12R-lipoxygenase (12R-LOX) or epidermal lipoxygenase-3 (eLOX3) is mutated. Deletion of 12R-LOX in mice results in impaired development of skin, which accounts for post-natal lethality. Conversely, overexpression of 12R-LOX has been implicated in Psoriasis, while deleterious mutations resulting in inactivation of 12R-LOX results in ichthyosis (skin disorder) characterized by disruption of cell-barrier functions. Thus, 12R-LOX forms a potential target for the development of proliferative skin disorders, including psoriasis.

### SUMMARY OF THE INVENTION

The present invention provides compounds, pharmaceutically acceptable salts or stereoisomers thereof and pharmaceutical compositions thereof. These compounds may be useful in therapies for treating LOX mediated diseases and/or disorders.

In one aspect, the present invention provides a compound of formula (I):

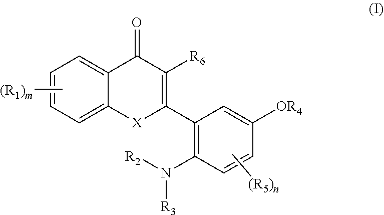

or a pharmaceutically acceptable salt or a stereoisomer thereof, wherein X, R₁, R₂, R₃, R₄, R₅, R₆, m and n are as defined in the detailed description infra.

In another aspect, the present invention provides a pharmaceutical composition comprising a therapeutically effective amount of a compound of formula (I) or a pharmaceutically acceptable salt or a stereoisomer thereof and one or more pharmaceutically acceptable carriers.

In another aspect, the present invention provides a pharmaceutical combination comprising a therapeutically effective amount of a compound of formula (I) or a pharmaceutically acceptable salt or a stereoisomer thereof and one or more therapeutically active agents.

In yet another aspect, the present invention provides a compound of formula (I) or a pharmaceutically acceptable salt or a stereoisomer thereof for use in the treatment of diseases and/or disorders mediated by aberrant or undesired expression of one or more lipoxygenases (LOXs).

### BRIEF DESCRIPTION OF ACCOMPANYING DRAWINGS

FIG. 1 illustrates Ni-NTA (nitrilotriacetic acid) purification of h-12R-LOX. A) SDS_PAGE (Sodium Dodecyl Sulfate Polyacrylamide Gel Electrophoresis) analysis of different eluted from Ni-Affinity column; B) Western blot analysis of Ni-NTA affinity based purified fractions.

FIG. 2 illustrates RP-HPLC (Reversed Phase-High Performance Liquid Chromatography) of the products formed by h12R-Lipoxygenase and auto oxidized products of methylated-arachidonic acid

### DETAILED DESCRIPTION OF THE INVENTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as the event or circumstance where the alkyl is not substituted.

“Halogen” refers to Fluorine, Chlorine, Bromine or Iodine.

The term “alkyl” refers to a straight or branched chain saturated aliphatic hydrocarbon that may be substituted or unsubstituted. In certain embodiments, the alkyl is C₁-C₆ alkyl. Examples of “alkyl” include but are not limited to methyl, ethyl, n-propyl, isopropyl, n-butyl, t-butyl, n-pentyl, isobutyl and the likes thereof.

The term “alkylsulfonyl” refers to a group —S(O₂)-alkyl, where alkyl is as defined above. Examples of “alkylsulfonyl” include, but are not limited to, methylsulfonyl and ethylsulfonyl, and propylsulfonyl.

The term “alkoxy” refers to a group —O-alkyl, wherein alkyl is as defined above. Representative examples include, but are not limited to, methoxy, ethoxy, propoxy, t-butoxy and the likes thereof.

The term “alkynyl” refers to an unsaturated hydrocarbon group which is linear or branched and has at least one carbon-carbon triple bond. In certain embodiments, an alkynyl group has 2 to 20 carbon atoms and in other embodiments, has 2 to 6 carbon atoms. An alkynyl group having 2 to 6 carbon atoms may be referred to as a —(C₂-C₆)alkynyl group. The alkynyl group may contain 1, 2 or 3 carbon-carbon triple bonds, or more. Preferably, alkynyl groups contain one or two triple bonds, most preferably one triple bond. In some instances, alkynyl moiety may be coupled to the remainder of the molecule through an alkyl linkage. Examples of alkynyl include, but are not limited to, ethynyl, 1-propynyl, 2-propynyl, 1-butynyl, 2-butynyl or 3-butynyl, 2-pentynyl, 3-pentynyl, 2-hexynyl, 3-hexynyl and the like.

The term “aryl” refers to optionally substituted unsaturated or partially saturated aromatic ring system having five to ten carbon atoms which are monocyclic, bicyclic or polycyclic and may optionally be replaced by one or more hetero atoms selected from N, O and S. Exemplary aryl groups include phenyl, naphthyl, indanyl, biphenyl and the likes thereof.

The term “arylalkyl” refers to an alkyl group substituted by one or more aryl groups, wherein the alkyl and aryl are same as defined above. Non-limiting examples of the arylalkyl group include phenylmethyl, phenylethyl, and the like.

The term “arylsulfonyl” refers to a group —S(O₂)-aryl, where aryl is as defined above. Examples of “arylsulfonyl” include, but are not limited to, phenylsulfonyl and tolylsulfonyl. The term “cycloalkyl” used herein, either alone or in combination with other radicals, denotes mono, bicyclic or polycyclic saturated, partially saturated hydrocarbon ring system of about 3 to 12 carbon atoms which may be substituted or unsubstituted. Exemplary “cycloalkyl” groups include, but are not limited to, cyclopropyl, cyclobutyl, cyclopentyl, cyclohexyl and cycloheptyl, perhydronaphthyl, adamantyl, noradamantyl and spirobicyclic groups such as spiro (4,4)non-2-yl.

The term “Heteroaryl” refers to monocyclic aromatic ring systems or fused bicyclic aromatic ring systems comprising two or more aromatic rings, preferably two to three ring systems. These heteroaryl rings contain one or more nitrogen, sulfur and/or oxygen atoms where N-oxides sulfur oxides and dioxides are permissible heteroatom substitutions. The term includes ring(s) optionally substituted with halogens, nitro, amino, alkoxy, alkyl sulfonyl amino, alkylcarbonylamino, carboxy, alkyl carbonyl, hydroxy, and alkyl. Examples of heteroaryl groups include furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, chromanyl, isochromanyl and the likes thereof.

The term “Heterocyclyl” refers to a stable 3 to 15 membered ring that is either saturated or has one or more degrees of unsaturation or unsaturated. These heterocyclic rings contain one or more heteroatoms selected from the group consisting of nitrogen sulfur and/or oxygen atoms where N-oxides, sulfur oxides and dioxides are permissible heteroatom substitutions. Such a ring is optionally fused to one or more of another heterocyclic ring(s), aryl ring(s) or cycloalkyl ring(s). Examples of such groups are selected from the group consisting of azetidinyl, acridinyl, pyrazolyl, imidazolyl, triazolyl, pyrrolyl, thiophenyl, thiazolyl, oxazolyl, isoxazolyl, furanyl, pyrazinyl, tetrahydroisoquinolinyl, piperidinyl, piperazinyl, morpholinyl, thiomorpholinyl, pyridazinyl, indolyl, isoindolyl, quinolinyl, chromanyl and the likes thereof. “Heterocyclylalkyl” refers to a heterocyclic ring radical defined above, directly bonded to an alkyl group. The heterocyclylalkyl radical is attached to the main structure at carbon atom in the alkyl group that results in the creation of a stable structure.

Unless otherwise specified, the term “substituted” as used herein refers to mono, bi, tri or tetra substitution with any one or more combination of the following substituents: hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted or unsubstituted heterocyclylalkyl ring, substituted or unsubstituted heteroarylalkyl, substituted or unsubstituted heterocyclic ring, substituted or unsubstituted guanidine, —COOR₈, —C(O) R₈, —C(S)R₈, —C(O)N(R₉)R₁₀, —C(O)ON(R₉)R₁₀, —NR₈CO(R₉)R₁₀, —N(R₈)SOR₉, —N(R₈)SO₂R₉, —(═N—N(R₉)R₁₀), —NR₈C(O)OR₉, —NR₉R₁₀, —NRC(O)R₉, —NR₈C(S)R₉, —NR₈C(S)NR₉R₁₀, —SONR₉R₁₀, —SO₂NR₉R₁₀, —OR₈, —OR₈C(O)NR₉R₁₀, —ORC(O)OR₉, —OC(O)R₈, —OC(O)NR₉R₁₀, —R₈NR₉C(O)R₁₀, —R₈OR₉, —R₈C(O)OR₉, —R₈C(O)NR₉R₁₀, —R₈C(O)R₉, —R₈OC(O)R₉, —SR₈, —SOR₈, —SO₂R₈, and —ONO₂, wherein R₈, R₉ and R₁₀ are independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted heterocyclylalkyl ring, substituted or unsubstituted heteroarylalkyl, or substituted or unsubstituted heterocyclic ring. Alternatively, R₉ and R₁₀ together with the nitrogen they are attached with, form a 4 to 8 membered ring which can be substituted or unsubstituted. According to one embodiment, the substituents in the aforementioned “substituted” groups cannot be further substituted. For example, when the substituent on “substituted alkyl” is “substituted aryl” the substituent on “substituted aryl” cannot be “substituted alkenyl”.

“Stereoisomers” refer to certain compounds described herein containing one or more chiral centres or may otherwise be capable of existing as multiple stereoisomers. Scope of the present invention includes pure stereoisomers as well as mixtures of stereoisomers such as purified enantiomers/diastereomers or enantiomerically/diastereomerically enriched mixtures.

“Bioisosteres” refers to compounds or groups that possess near molecular shapes and volumes, approximately the same distribution of electrons and which exhibit similar physical properties such as hydrophobicity. Bioisosteric compounds affect the same biochemically associated systems as agonist or antagonists and thereby produce biological properties that are related to each other.

“Pharmaceutically acceptable salts” forming part of this invention include salts derived from inorganic bases such as Li, Na, K, Ca, Mg, Fe, Cu, Zn, Al, Mn; salts of organic bases such as N,N′-diacetylethylenediamine, 2-dimethylaminoethanol, isopropylamine, morpholine, piperazine, piperidine, procaine, diethylamine, triethylamine, trimethylamine, tripropylamine, tromethamine, choline hydroxide, dicyclohexylamine, metformin, benzylamine, phenylethylamine, dialkylamine, trialkylamine, thiamine, aminopyrimidine, aminopyridine, purine, pyrimidine, spermidine, and the like; chiral bases like alkylphenylamine, glycinol, phenyl glycinol and the like, salts of natural amino acids such as glycine, alanine, valine, leucine, isoleucine, lysine, arginine, serine, threonine, phenylalanine; unnatural amino acids such as D-isomers or substituted amino acids; salts of acidic amino acids such as aspartic acid, glutamic acid; guanidine, substituted guanidine wherein the substituents are selected from nitro, amino, alkyl, alkenyl, alkynyl, ammonium or substituted ammonium salts. Salts may include acid addition salts where appropriate which are sulphates, nitrates, phosphates, perchlorates, borates, hydrohalides, acetates, tartrates, maleates, citrates, succinates, methanesulfonates, benzoates, salicylates, hydroxynaphthoates, benzenesulfonates, ascorbates and the likes thereof.

“Pharmaceutically acceptable solvates” may be hydrates or comprising other solvents of crystallization such as alcohols.

“Compounds of the present invention” or “present invention” or “compounds of the disclosure” or “compound of the disclosure” refers to compounds of formula (I), (IA), (IB), (IC), (ID) and/or (IE) as herein defined, their derivatives, their analogs, their tautomeric forms, their stereoisomers, their bioisosteres, their diastereomers, their polymorphs, their enantiomers, their appropriate N-oxides, their pharmaceutically acceptable salts, their pharmaceutically acceptable hydrates, their pharmaceutically acceptable solvates and pharmaceutically acceptable compositions containing them.

The stereoisomers of the compounds forming part of this invention may be prepared by using reactants in their single enantiomeric form in the process wherever possible or by conducting the reaction in the presence of reagents or catalysts in their single enantiomer form or by resolving the mixture of stereoisomers by conventional methods. Some of the preferred methods include use of microbial resolution, resolving the diastereomeric salts formed with chiral acids such as mandelic acid, camphorsulfonic acid, tartaric acid, lactic acid, and the like wherever applicable or chiral bases such as brucine, cinchona alkaloids and their derivatives and the like. Different polymorphs of a compound of formula (I), (IA), (IB), (IC), (ID) and/or (IE) of present invention may be prepared by crystallization of the corresponding compounds of (I), (IA), (IB), (IC), (ID) and/or (IE) under different conditions. For example, making use of commonly used solvents or their mixtures for recrystallization, crystallization at different temperature ranges, different cooling techniques like very fast to very slow cooling during crystallization procedure, by exposing to room temp, by heating or melting the compound followed by gradual cooling and the like. The presence of polymorphs may be determined by one or more methods like solid probe NMR spectroscopy, DSC, TGA, Powder X-Ray diffraction and IR.

The present invention also provides pharmaceutical compositions containing the compounds of invention as defined above, their derivatives, analogs, tautomeric forms, stereoisomers, bioisosteres, polymorphs, enantiomers, diastereomers, their pharmaceutically acceptable salts or solvates in combination with suitable pharmaceutically acceptable carriers, and/or diluents.

The pharmaceutical compositions according to the present invention are useful as anti-psoriatic agents. The pharmaceutical composition may be tablets, capsules, powders, syrups, solutions, suspensions, sprays and the likes thereof and may contain flavorants, sweeteners etc., in a suitable solid or liquid carriers or diluents or in a suitable sterile media to form injectable solutions or suspensions.

The invention also encompasses prodrugs of compounds of the invention, which on administration undergo chemical conversion by metabolic processes before becoming active pharmacological substances. In general, such prodrugs will be functional derivatives of compounds of invention, which are readily convertible in vivo into compounds of the invention. The invention also encompasses the active metabolites of the compounds of the present invention of general Formula (I).

### Prodrugs

In certain embodiments, the compounds can be formulated and administered in a prodrug form. In general, prodrugs comprise functional derivatives of the claimed compounds, which are capable of being enzymatically activated or converted into the more active parent form. Thus, in the treatment methods of the present invention, the term “administering” encompasses the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in Wihnan, 14 Biochem. Soc. Trans. 375-82 (1986); Stella et al., Prodrugs: A Chemical Approach to Targeted Drug Delivery in Directed Drug Delivery 247-67 (1985).

Each embodiment is provided by way of explanation of the invention and not by way of limitation of the invention. In fact, it will be apparent to those skilled in the art that various modifications and variations can be made to the compounds, compositions and methods described herein without departing from the scope or spirit of the invention. For instance, features illustrated or described as part of one embodiment can be applied to another embodiment to yield a still further embodiment. Thus, it is intended that the present invention includes such modifications and variations and their equivalents. Other objects, features and aspects of the present invention are disclosed in or are obvious from, the following detailed description. It is to be understood by one of ordinary skill in the art that the present discussion is a description of exemplary embodiments only and is not to be construed as limiting the broader aspects of the present invention.

In an embodiment, the present invention provides a compound of formula (I):

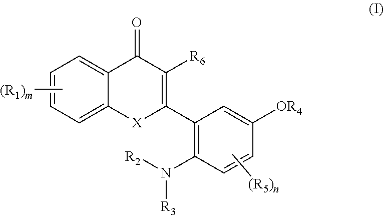

or a pharmaceutically acceptable salt or a stereoisomer thereof, wherein,

each of R₁, R₅ and R₆, at each occurrence, is independently selected from hydrogen, halogen, optionally substituted amine, optionally substituted alkyl, optionally substituted alkenyl, optionally substituted alkynyl, optionally substituted alkoxyalkyl, optionally substituted alkoxy, optionally substituted cycloalkyl, optionally substituted acyl, optionally substituted alkylsulfonyl, optionally substituted arylsulfonyl, optionally substituted heteroarylsulfonyl, optionally substituted aryl, —OR, and —COOR;

R₂ and R₃ are independently selected from hydrogen, optionally substituted alkyl, optionally substituted alkenyl, optionally substituted alkynyl, optionally substituted cycloalkyl, optionally substituted alkylsulfonyl, optionally substituted arylsulfonyl, optionally substituted heteroarylsulfonyl, optionally substituted aryl, optionally substituted arylalkyl, —CO-(optionally substituted alkyl), —CO-(optionally substituted aryl), optionally substituted heterocyclyl and optionally substituted heteroaryl, wherein, the optional substituent, at each occurrence, is independently selected from one or more halogen, hydroxyl, alkyl, alkoxyalkyl, haloalkyl, alkoxy, nitro, cycloalkyl, alkylsulfonyl, arylsulfonyl, and —COOR; or

R₂ and R₃ together with the nitrogen to which they attached form an imine of formula, —N═C(R₂ₐ)(R_{2b}), wherein R₂ₐ and R_{2b} are independently selected from hydrogen, alkyl and aryl;

R₄ is selected from hydrogen, optionally substituted alkyl, optionally substituted alkenyl, optionally substituted alkynyl, optionally substituted alkoxyalkyl, optionally substituted cycloalkyl, optionally substituted aryl, substituted alkylsulfonyl and optionally substituted arylsulfonyl;

R is hydrogen, optionally substituted alkyl or optionally substituted aryl;

X is oxygen, sulfur or NR₇; wherein R₇ is hydrogen, halogen, hydroxyl, optionally substituted alkyl, optionally substituted alkoxyalkyl, optionally substituted alkoxy, optionally substituted cycloalkyl, optionally substituted acyl, optionally substituted alkylsulfonyl, optionally substituted arylsulfonyl, optionally substituted heteroarylsulfonyl, optionally substituted aryl, optionally substituted heterocyclyl and optionally substituted heteroaryl;

m is 1, 2, 3, or 4; and

n is 1, 2 or 3.

In certain embodiments, the present invention provides a compound of formula (IA):

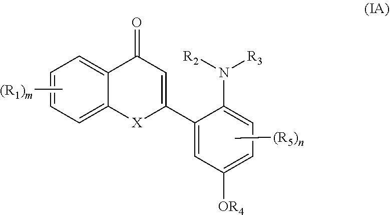

or a pharmaceutically acceptable salt or a stereoisomer thereof, wherein R₁, R₂, R₃, R₄, R₅, X, m and n are same as defined in formula (I).

In certain embodiments, the present invention provides a compound of formula (IB):

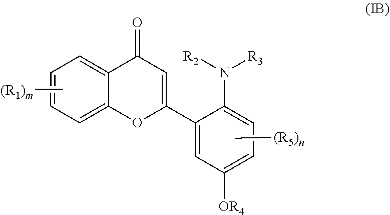

or a pharmaceutically acceptable salt or a stereoisomer thereof, wherein, R₁, R₂, R₃, R₄, R₅, m and n are same as defined in formula (I).

In certain embodiments, the present invention provides compounds of formula (I), (IA) and/or (IB), wherein m is 1 or 2.

In certain embodiments, the present invention provides compounds of formula (I), (IA) and/or (IB), wherein n is 1.

In certain embodiments, the present invention provides compounds of formula (I), (IA) and/or (IB), wherein m is 1 and n is 1.

In certain embodiments, the present invention provides compounds of formula (I), (IA) and/or (IB); wherein R₁, at each occurrence, is independently selected from hydrogen, halogen, —OR, optionally substituted alkyl, optionally substituted alkoxyalkyl, and optionally substituted alkoxy. R is same as defined in formula (I).

In certain embodiments, the present invention provides compounds of formula (I), (IA) and/or (IB); wherein R₅, at each occurrence, is independently selected from hydrogen, halogen, optionally substituted alkyl, and optionally substituted alkoxy. In further embodiments, R₅, at each occurrence, is independently hydrogen or halogen.

In certain embodiments, the present invention provides a compound of formula (IC):

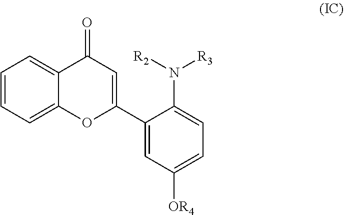

or a pharmaceutically acceptable salt or a stereoisomer thereof, wherein R₂, R₃, and R₄ are same as defined in formula (I).

In certain embodiments, the present invention provides a compound of formula (ID):

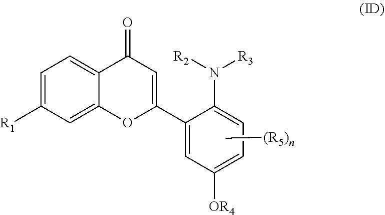

or a pharmaceutically acceptable salt or a stereoisomer thereof, wherein R₁, R₂, R₃, R₄, R₅ and n are same as defined in formula (I).

In certain embodiments, the present invention provides a compound of formula (IE):

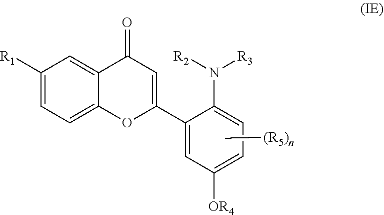

or a pharmaceutically acceptable salt or a stereoisomer thereof, wherein, R₁, R₂, R₃, R₄, R₅ and n are same as defined in formula (I).

In certain embodiments, R₁ in formula (I), (IA), (IB), (IC), (ID) and/or (IE) is selected from hydrogen, halogen or hydroxyl.

In certain embodiments, the present invention provides compounds of formula (I), (IA), (IB), (IC), (ID) and/or (IE), wherein R₂ and R₃ are independently selected from hydrogen, optionally substituted alkylsulfonyl, optionally substituted arylsulfonyl, and CO-aryl, wherein aryl of —CO-aryl, at each occurrence, is optionally substituted with one or more groups selected from halogen, hydroxyl, alkyl, alkoxyalkyl, alkoxy, cycloalkyl, alkylsulfonyl, arylsulfonyl, and —COOR, wherein R is hydrogen, alkyl or aryl. In further embodiments, R₂ is hydrogen and R₃ is hydrogen, alkylsulfonyl, arylsulfonyl, or CO-aryl, wherein aryl, at each occurrence, is optionally substituted with one or more groups selected from halogen, hydroxyl, nitro and alkyl. In certain embodiments, R₂ is hydrogen and R₃ is arylsulfonyl optionally substituted with halogen, hydroxyl and alkyl. In some instances, R₃ is phenylsulfonyl optionally substituted with alkyl or nitro. In further instances, R₃ is phenylsulfonyl substituted with methyl.

In certain embodiments, the present invention provides compounds of formula (I), (IA), (IB), (IC), (ID) and/or (IE); wherein R₂ and R₃ are hydrogen.

In certain embodiments, the present invention provides compounds of formula (I), (IA), (IB), (IC), (ID) and/or (IE); wherein R₂ is hydrogen and R₃ is alkylsulfonyl. In further embodiments, R₃ is methylsulfonyl or ethylsulfonyl.

In certain embodiments, the present invention provides compounds of formula (I), (IA), (IB), (IC), (ID) and/or (IE); wherein R₂ is hydrogen and R₃ is arylsulfonyl, wherein aryl of the arylsulfonyl, at each occurrence, is optionally substituted with one or more halogen, hydroxyl, nitro and alkyl. In further embodiments, R₃ is phenylsulfonyl optionally substituted with alkyl or nitro. In some instances, R₃ is phenylsulfonyl or phenylsulfonyl substituted with methyl or nitro.

In certain embodiments, the present invention provides compounds of formula (I), (IA), (IB), (IC), (ID) and/or (IE); wherein R₂ is hydrogen and R₃ is —CO-aryl. In further embodiments, R₃ is-CO-phenyl.

In certain embodiments, the present invention provides compounds of formula (I), (IA), (IB), (IC), (ID) and/or (IE); wherein R₄ is selected from hydrogen, alkyl, alkynyl and alkylsulfonyl.

In certain embodiments, the present invention provides compounds of formula (I), (IA), (IB), (IC), (ID) and/or (IE); wherein R₄ is selected from hydrogen, methyl, ethyl, propargyl and methylsulfonyl. In further embodiments, R₄ is hydrogen or propargyl.

In certain embodiments, the compounds of the present invention can also contain unnatural proportions of atomic isotopes at one or more of the atoms that constitute such compounds. For example, the present invention also embraces isotopically-labeled variants of the present invention which are identical to those recited herein, but for the fact that one or more atoms of the compound are replaced by an atom having the atomic mass or mass number different from the predominant atomic mass or mass number usually found in nature for the atom. All isotopes of any particular atom or element as specified are contemplated within the scope of the compounds of the invention and their uses. Exemplary isotopes that can be incorporated in to compounds of the invention include isotopes of hydrogen, carbon, nitrogen, oxygen, phosphorous, sulfur, fluorine, chlorine and iodine, such as ²H (“D”), ³H, ⁿC, ¹³C, ¹⁴C, ¹³N, ¹⁵N, ¹⁵O, ¹⁷O, ¹⁸O, ³⁵S, ¹⁸F, ³⁶Cl, ¹²³I and ¹²⁵I. Isotopically labeled compounds of the present inventions can generally be prepared by following procedures analogous to those disclosed in the schemes and/or in the examples herein below, by substituting an isotopically labeled reagent for a non-isotopically labeled reagent.

In certain embodiments, the present invention provides a compound selected from:
2-(2-Amino-5-hydroxyphenyl)-4H-chromen-4-one;
4-(Methylsulfonamido)-3-(4-oxo-4H-chromen-2-yl)phenyl methanesulfonate;
2-(2-Amino-5-hydroxy-4-iodophenyl)-4H-chromen-4-one;
2-(2-Amino-5-methoxyphenyl)-4H-chromen-4-one;
2-(2-Amino-5-ethoxyphenyl)-4H-chromen-4-one;
2-(2-Amino-5-(prop-2-ynyloxy)phenyl)-4H-chromen-4-one;
N-(2-(4-Oxo-4H-chromen-2-yl)-4-(prop-2-ynyloxy)phenyl)benzenesulfonamide;
N-(4-Hydroxy-2-(4-oxo-4H-chromen-2-yl)phenyl)benzenesulfonamide;
4-Methyl-N-(2-(4-oxo-4H-chromen-2-yl)-4-(prop-2-ynyloxy)phenyl)benzenesulfonamide;
N-(4-Hydroxy-2-(4-oxo-4H-chromen-2-yl)phenyl)-4-methylbenzenesulfonamide;
N-(2-(4-Oxo-4H-chromen-2-yl)-4-(prop-2-ynyloxy)phenyl)benzamide;
N-(4-Hydroxy-2-(4-oxo-4H-chromen-2-yl)phenyl)benzamide;
2-(2-Amino-5-hydroxyphenyl)-7-fluoro-4H-chromen-4-one;
2-(2-Amino-5-hydroxyphenyl)-6-bromo-4H-chromen-4-one;
N-(4-Hydroxy-2-(4-oxo-4H-chromen-2-yl)phenyl)-4-nitrobenzenesulfonamide;
2-(2-Amino-5-hydroxyphenyl)-6-hydroxy-4H-chromen-4-one;
N-(4-Hydroxy-2-(4-oxo-4H-chromen-2-yl)phenyl)-4-(trifluoromethyl)benzenesulfonamide; and
2-(2-Amino-5-hydroxyphenyl)-7-bromo-4H-chromen-4-one;
   or a pharmaceutically acceptable salt or a stereoisomer thereof.

In certain embodiments, the present invention provides a pharmaceutical composition comprising a compound of formula (I), (IA), (IB), (IC), (ID) and/or (IE), optionally admixed with a pharmaceutically acceptable carrier or diluent.

The compounds of formula (I), (IA), (IB), (IC), (ID) and/or (IE), or a pharmaceutically acceptable salt or a stereoisomer thereof may be useful in the treatment of diseases and/or disorders mediated by aberrant or undesired expression of one or more lipoxygenases (LOXs). The LOX mediated disease and/or disorder is selected from a group comprising Alzheimer's disease, cancer, cardiovascular disease, diabetes (type 1 and/or type 2), diabetic kidney disease, diabetic nerve disease, heparin induced thrombocytopenia, non-alcoholic steatohepatitis, platelet hemostasis, skin diseases (such as psoriasis), thrombosis, asthma, arthritis, ulcerative colitis, allergic diseases, auto-immune diseases, Parkinson's disease, atherosclerosis, hypertension, Schizophrenia and sepsis.

The present invention also provides methods for formulating the disclosed compounds for pharmaceutical administration.

The compositions and methods of the present invention may be utilized to treat a subject in need thereof. In certain embodiments, the subject is a mammal such as a human or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol and oils such as olive oil or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, and lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation of pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin or as an eye drop). The compound may also be formulated for inhalation. In yet another embodiment, a compound may be simply dissolved or suspended in sterile water.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers or finely divided solid carriers or both and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules or as a solution or a suspension in an aqueous or non-aqueous liquid or as an oil-in-water or water-in-oil liquid emulsion or as an elixir or syrup or as pastilles (using an inert base, such as gelatin and glycerin or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate and/or any of the following: (1) fillers or extenders, such as starches, lactose, sucrose, glucose, mannitol and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter or by incorporating sterilizing agents in the form of sterile solid compositions that can be dissolved in sterile water or some other sterile injectable medium immediately before use. These compositions may also optionally contain opacifying agents and may be of a composition that they release the active ingredient(s) only or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Liquid dosage forms useful for oral administration include pharmaceutically acceptable emulsions, lyophiles for reconstitution, microemulsions, suspensions, solutions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth and mixtures thereof.

Formulations of the pharmaceutical compositions for rectal, vaginal or urethral administration may be presented as a suppository, which may be prepared by mixing one or more active compounds with one or more suitable nonirritating excipients or carriers comprising, for example, cocoa butter, polyethylene glycol, a suppository wax or a salicylate and which is solid at room temperature, but liquid at body temperature and, therefore, will melt in the rectum or vaginal cavity and release the active compound.

Formulations of the pharmaceutical compositions for administration to the mouth may be presented as a mouthwash or an oral spray or an oral ointment.

Alternatively, or additionally, compositions can be formulated for delivery via a catheter, stent, wire or other intraluminal device. Delivery via such devices may be especially useful for delivery to the bladder, urethra, ureter, rectum or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier and with any preservatives, buffers or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide or mixtures thereof.

Powders and sprays can contain, in addition to an active compound, excipients such as lactose, talc, silicic acid, aluminum hydroxide, calcium silicates and polyamide powder or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. If desired, liquid ophthalmic formulations have properties similar to that of lacrimal fluids, aqueous humor or vitreous humor or are compatable with such fluids. A preferred route of administration is local administration (e.g., topical administration, such as eye drops or administration via an implant).

The phrases “parenteral administration” and “administered parenterally” as used herein means modes of administration other than enteral and topical administration, usually by injection and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol and the like) and suitable mixtures thereof, vegetable oils, such as olive oil and injectable organic esters, such as ethyl oleate. Proper fluidity can be maintained, for example, by the use of coating materials, such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid and the like. It may also be desirable to include isotonic agents, such as sugars, sodium chloride and the like into the compositions. In addition, prolonged absorption of the injectable pharmaceutical form may be brought about by the inclusion of agents that delay absorption such as aluminium monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition and mode of administration, without being toxic to the patient.

The selected dosage level will depend upon a variety of factors including the activity of the particular compound or combination of compounds employed or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age and medical history of the subject. Other factors which influence the effective amount may include, but are not limited to, the severity of the patient's condition, the disorder being treated, the stability of the compound and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In yet another embodiment of the present invention, the active compound may be administered two or three times daily. In preferred embodiments, the active compound will be administered once daily.

The patient receiving this treatment is any animal in need, including primates, in particular humans and other mammals such as equines, cattle, swine and sheep; and poultry and pets in general.

Wetting agents, emulsifiers and lubricants, such as sodium lauryl sulfate and magnesium stearate, as well as coloring agents, coating agents, release agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol and the like; and (3) metal-chelating agents, such as citric acid, ethylenediamine tetraacetic acid (EDTA), sorbitol, tartaric acid, phosphoric acid and the like.

The present invention further provides use of a compound of the present invention for the preparation of a medicament.

In certain embodiments, compounds of the present invention inhibit one or more Lipoxygenases (LOXs) such as 5-LOX, 12-LOX and 15-LOX. In some embodiments, compounds of the present invention may be selective inhibitors. For example, compound that is selective for 12-LOX may not significantly affect the activity of other LOXs such as 5-LOX and 15-LOX.

The invention further provides a method for treating or preventing a disease and/or disorder mediated by aberrant or undesired expression of one or more LOXs. The method comprises administering a therapeutically or prophylactically effective amount of a compound of the present invention to a subject in need thereof.

In certain embodiments, LOX mediated diseases and/or disorders include those where LOX is a direct mediator of the diseases and/or disorders, as well as those where the inhibition of LOX results in therapeutic value in the treatment or prevention of the diseases and/or disorders.

In certain embodiments, LOX is 5-LOX, 12S/12R-LOX and/or 15-LOX. In some instances, the LOX is 12S/12R-LOX. In further embodiments, the LOX is 12R-LOX.

In certain embodiments, the LOX mediated disease and/or disorder is selected from a group comprising Alzheimer's disease, cancer, cardiovascular disease, diabetes (type 1 and/or type 2), diabetic kidney disease, diabetic nerve disease, heparin induced thrombocytopenia, non-alcoholic steatohepatitis, platelet hemostasis, skin diseases, thrombosis, asthma, arthritis, ulcerative colitis, allergic diseases, auto-immune diseases, Parkinson's disease, atherosclerosis, hypertension, Schizophrenia and sepsis.

Examples of skin diseases include, but are not limited to ichthyosis, psoriasis, skin cancers, autosomal recessive congenital ichthyosis (ARCI) and systematic sclerosis.

In certain embodiment, the present invention provides methods for treating psoriasis and other skin related diseases, wherein the method comprises administering a therapeutically effective amount of a compound of the present invention to a subject in need thereof.

The compounds of the present invention may be used as single drugs (monotherapy) or conjointly with one or more other agents (conjoint therapy). The compounds may be used by themselves or, preferably, in a pharmaceutical composition in which the compound is mixed with one or more pharmaceutically acceptable materials.

The dosage of the compounds of the present invention varies depending on a patient's age, weight or symptoms, as well as the compound's potency or therapeutic efficacy, the dosing regimen and/or treatment time. Generally, suitable routes of administration may, for example, include oral, eyedrop, rectal, transmucosal, topical or intestinal administration; parenteral delivery, including intramuscular, subcutaneous, intramedullary injections, as well as intrathecal, direct intraventricular, intravenous, intraperitoneal, intranasal or intraocular injections. The compounds of the invention may be administered in an amount of 0.5 mg or 1 mg up to 500 mg, 1 g or 2 g per dosage regimen. The dosage may be administered once per week, once per three days, once per two days, once per day, twice per day, three times per day or more often. In alternative embodiments, in certain adults the compound can be continuously administered by intravenous administration for a period of time designated by a physician. Since the dosage is affected by various conditions, an amount less than or greater than the dosage ranges contemplated about may be implemented in certain cases. A physician can readily determine the appropriate dosage for a patient undergoing therapeutic treatment.

The compounds of the present invention may be administered in combination with one or more other drugs (1) to complement and/or enhance effect of the compound of the present invention, (2) to modulate pharmacodynamics, improve absorption or reduce dosage of the compound of the present invention and/or (3) to reduce or ameliorate the side effects of the compound of the present invention. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a 24 separate formulation, either concomitantly or sequentially. In yet another embodiment, the different therapeutic compounds can be administered within 1 h, 12 h, 24 h, 36 h, 48 h, 72 h or a week of one another. Thus, an individual who receives such treatment can benefit from a combined effect of different therapeutic compounds. The respective compounds may be administered by the same or different route and the same or different method.

The dosage of the other drug can be a dosage that has been clinically used or may be a reduced dosage that is effective when administered in combination with a compound of the present invention. The ratio of the compound of the present invention and the other drug can vary according to age and weight of a subject to be administered, administration method, administration time, disorder to be treated, symptom and combination thereof. For example, the other drug may be used in an amount of 0.01 to 100 parts by mass, based on 1 part by mass of the compound of the present invention.

Conjoint therapy can be employed to treat any diseases discussed herein. For example, in the methods of the invention directed to the treatment of LOX (such as 5-LOX, 12-LOX and 15-LOX) mediated diseases and/or disorders, the compound of the present invention can be used with an existing LOX inhibitor(s) conjointly using a single pharmaceutical composition or a combination of different pharmaceutical compositions. Examples of the LOX inhibitor(s) include Zileuton and other LOX inhibitors.

In certain embodiments, different compounds of the invention may be conjointly administered with one or more other compounds of the invention. Moreover, such combinations may be conjointly administered with other therapeutic agents, such as other agents suitable for the treatment of diseases and/or disorders listed above. In a further embodiment, the other therapeutic agents include anti-inflammatory drugs such as NSAIDs, COX-2 specific inhibitors (e.g., Celebrex and Bextra) and COX-2/5-LOX dual inhibitors.

The present invention also provides a process for preparation of the compounds of general formula (I) are set forth in the below Examples and generalized Scheme. One of skill in the art will recognize that scheme can be adapted to produce the compounds of general formula (I) and pharmaceutically acceptable salts or stereo isomers of compounds of general formula (I) according to the present invention.

The schemes are given for the purpose of illustrating the invention, and are not intended to limit the scope or spirit of the invention. Starting materials shown in the schemes can be obtained from commercial sources or prepared based on procedures described in the literature. Furthermore, in the following schemes, where specific acids, bases, reagents, coupling agents, solvents, etc. are mentioned, it is understood that other suitable acids, bases, reagents, coupling agents etc. may be used and are included within the scope of the present invention. Modifications to reaction conditions, for example, temperature, duration of the reaction or combinations thereof, are envisioned as part of the present invention. All possible stereoisomers are envisioned within the scope of this invention.

The intermediates required for the synthesis are commercially available or alternatively, these intermediates can be prepared using known literature methods. The invention is described in greater detail by way of specific examples.

It is contemplated that some of the intermediates disclosed in the present invention are used for the next step without any characterization data. It is meant to be understood that the order of the steps in the processes may be varied, that reagents, solvents and reaction conditions may be substituted for those specifically mentioned, and that vulnerable moieties may be protected and protected, as necessary. The variables “X”, “R₁”, “R₂”, “R₃”, “R₄”, “R₅”, “m” and “n” independently represents all the possible substitutions as disclosed in compound of formula (I).

### General Synthetic Scheme(s):

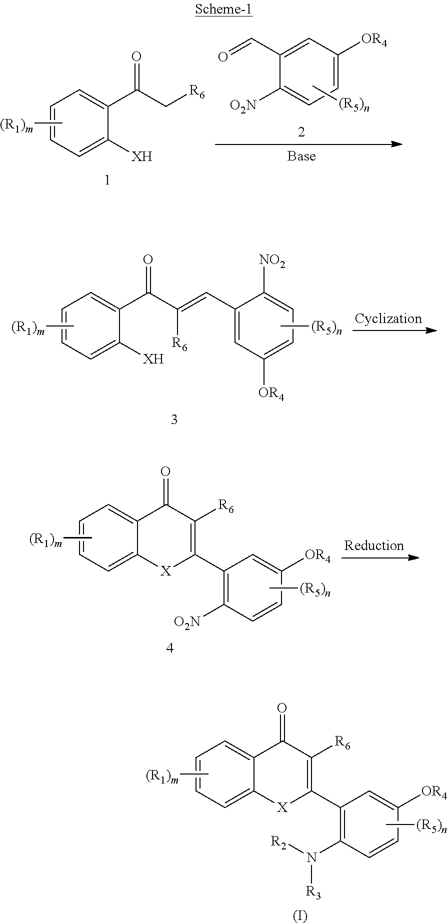

Some of the compounds of the present invention are prepared by reacting a compound of formula 1 with a compound of formula 2 using a suitable base selected from KOH, NaOH, K₂CO₃, Na₂CO₃, Cs₂CO₃, NaHCO₃, DBU, piperidine, Et₃N, DIPEA, DABCO, NaH and the likes in the absence or presence of a suitable solvent selected from DMSO, DMF, MeOH, EtOH, i-PrOH, benzene, toluene, xylene, THF, 1,4-dioxane, diethyl ether, diphenyl ether, and the likes thereof to give compound of formula 3 which is then cyclized in presence of suitable catalyst such as CuI, CuBr, CuCl and iodine, and suitable solvent selected from DMSO, DMF, MeOH, EtOH, i-PrOH, acetonitrile, THF, 1,4-dioxane, diethyl ether, diphenyl ether, dichloromethane, chloroform, 1,2-dichloroethane or the likes thereof to give compound 4. The compound 4 is then reduced in presence of a suitable reducing agent such as Fe/H⁺, Sn/H⁺, catalytic hydrogenation (e.g. H₂/Pd, H₂/Raney Ni), LiAlH₄, NaBH₄, Zn/HCl, Zn/NH₄Cl to give a compound of formula (I).

It is understood that in any of the above schemes, any reactive group in the substrate molecule may be protected according to any conventional procedure known in the prior art. Suitable protecting groups comprise N-Boc, N-Cbz, N-Fmoc, alkyl, benzophenone imine for protection of amino group, acetal protection for aldehyde, ketal protection for ketone.

### EXAMPLES

The following examples are given by way of illustration of the working of the invention in actual practice and therefore should not be construed to limit the scope of present invention.

### General Method(s):

Unless stated otherwise, reactions were performed under nitrogen atmosphere. Reactions were monitored by thin layer chromatography (TLC) on silica gel plates (60 F254), visualizing with ultraviolet light or iodine spray. Flash chromatography was performed on silica gel (100-200 mesh) using distilled hexane, ethyl acetate, dichloromethane and methanol. ¹H and ¹³C NMR spectra were recorded either in CDCl₃, DMSO-d₆ or CF₃CO₂H solution by using 400 and 100 MHz spectrometers, respectively. Proton chemical shifts (δ) are relative to tetramethylsilane (TMS, δ=0.00) as internal standard and expressed in ppm. Spin multiplicities are given as s (singlet), d (doublet), t (triplet) and m (multiplet) as well as b (broad). Coupling constants (J) are given in hertz. Melting points were determined using melting point apparatus and are uncorrected. MS spectra were obtained by using a mass spectrometer.

### Example 1: 2-(2-Amino-5-hydroxyphenyl)-4H-chromen-4-one (Compound 1)

### Step 1: Preparation of (E)-3-(5-hydroxy-2-nitrophenyl)-1-(2-hydroxyphenyl) prop-2-en-1-one (3)

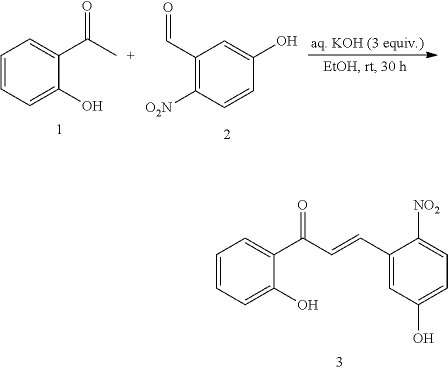

To a solution of intermediate 1 (0.136 g, 1 mmol) in EtOH (20 mL), was added an aqueous solution of KOH (0.168 g, 3 mmol, 10 mL). Then the resultant solution was left for stirring at ambient temperature for 30 minutes. Intermediate 2 (0.167 g, 1 mmol) was then added and the mixture was stirred at ambient temperature for 30 h. After completion of reaction, excess solvent was evaporated under vacuum, excess KOH was neutralized by using aqueous HCl (brought to pH˜6) and then the reaction mixture was extracted with ethyl acetate (3×30 mL). Organic layers were combined, dried over anhydrous Na₂SO₄, filtered, and evaporated under low vacuum. The residue was then purified using column chromatography (30% EtOAc/n-hexane) to give the intermediate 3.

Yield=60%; Yellow solid; R_{f}=0.23 (30% EtOAc/n-hexane); ¹H NMR (400 MHz, CDCl₃) δ: 11.89 (s, 1H, OH, D₂O exch.), 10.91 (s, 1H, OH, D₂O exch.), 7.99 (d, J=8.9 Hz, 1H, ═CH), 7.93-7.90 (m, 1H, ArH), 7.53-7.49 (m, 1H, ArH), 7.32 (d, J=2.6, 1H, ArH), 6.97-6.91 (m, 2H, ArH and ═CH), 6.82 (dd, J=8.9 & 2.7, Hz, 1H, ArH), 5.83 (d, J=7.9 Hz, 1H, ArH), 5.68 (s, 1H, ArH); MS (ES mass): 286.0 (M+1).

### Step 2: Preparation of 2-(5-hydroxy-2-nitrophenyl)-4H-chromen-4-one (4)

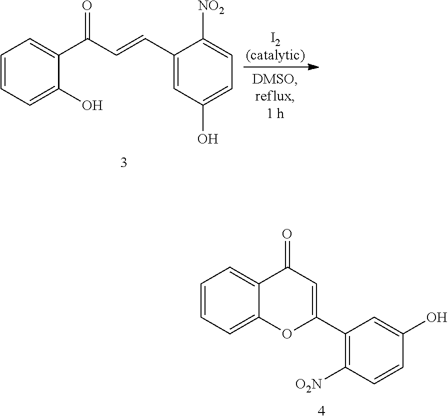 [SMILES: O=C(/C=C/c1cc(O)ccc1[N+](=O)[O-])c1ccccc1O ; O=c1cc(-c2cc(O)ccc2[N+](=O)[O-])oc2ccccc12]

Intermediate 3 (0.285 g, 1 mmol) and a catalytic amount of I₂ were dissolved in DMSO (10 mL) and refluxed at 200° C. for 1 h. After completion of reaction (monitored by TLC), the temperature of the reaction mixture was brought down to room temperature and water (50 mL) was added. The mixture was extracted with EtOAc (3×30 mL). The organic layers were collected, combined, washed with water (3×20 mL), dried over anhydrous sodium sulfate, filtered and concentrated in vacuo. The residue obtained was purified by column chromatography using 40% of ethyl acetate/n-hexane as an eluent to give the titled compound.

Yield=95%; Pale yellow solid; mp: 262-264° C.; R_{f}=0.21 (40% EtOAc/n-hexane); ¹H NMR (400 MHz, CDCl₃) δ: 11.45 (s, 1H, OH, D₂O exch.), 8.16 (d, J=8.7 Hz, 1H, ArH), 8.08 (dd, J=7.9 & 1.5 Hz, 1H, ArH), 7.83-7.84 (m, 1H, ArH), 7.57-7.50 (m, 2H, ArH), 7.14-7.09 (m, 2H, ArH), 6.64 (s, 1H, ═CH); MS (ES mass): 284.0 (M+1); HPLC: 96.6%; Column: XBridge C-18 150*4.6 mm, 3.5 μm; Mobile phase: A) 0.1% TFA in water, B) ACN; (T/B %): 0/10, 3/10, 15/95, 23/95, 25/10, 30/10; Flow rate: 1.0 mL/min; Diluent: ACN:Water (80:20); UV: 230.0 nm; Retention time: 11.3 min.

### Step 3: Preparation of 2-(2-amino-5-hydroxyphenyl)-4H-chromen-4-one

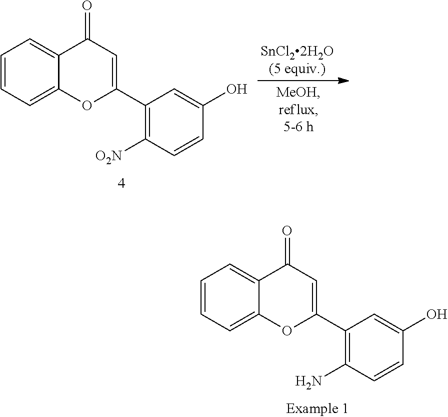 [SMILES: O=c1cc(-c2cc(O)ccc2[N+](=O)[O-])oc2ccccc12 ; Nc1ccc(O)cc1-c1cc(=O)c2ccccc2o1]

To a solution of intermediate 4 (0.283, 1 mmol) in MeOH was added SnCl₂.2H₂O (1.13 g, 5 mmol). Then the reaction mixture was refluxed until disappearance of the starting material (monitored by TLC). Then ice-cold water was added to the reaction mixture and the residue obtained was diluted with 2N NaOH (50 mL) solution. The aqueous layer was then extracted with EtOAc (3×50 mL). The organic layers were collected, combined, dried over anhydrous Na₂SO₄, filtered and concentrated. The crude product was then purified by column chromatography using 40% of EtOAc/n-hexane as eluent to yield the compound 1.

Yield=85%; Brick red solid; mp: 202-204° C.; R_{f}=0.19 (40% EtOAc/n-hexane); ¹H NMR (400 MHz, CDCl₃) δ: 8.82 (s, 1H, OH, D₂O exch.), 8.03 (dd, J=7.9 & 1.4 Hz, 1H, ArH), 7.81-7.77 (m, 1H, ArH), 7.70-7.68 (m, 1H, ArH), 7.49-7.45 (m, 1H, ArH), 6.85 (d, J=2.5 Hz, 1H, ArH), 6.75-6.69 (m, 2H, ArH), 6.54 (s, 1H, ═CH), 5.07 (s, 2H, NH₂, D₂O exch.); MS (ES mass): 254.1 (M+1); HPLC: 99.8%; Column: XBridge C-18 150*4.6 mm, 3.5 μm; Mobile phase: A) 0.1% TFA in water, B) ACN; (T/B %): 0/10, 3/10, 15/95, 23/95, 25/10, 30/10; Flow rate: 1.0 mL/min; Diluent: ACN:Water (80:20); UV: 240.0 nm; Retention time: 7.4 min.

### Example 2: 4-(Methylsulfonamido)-3-(4-oxo-4H-chromen-2-yl)phenylmethane Sulfonate (Compound 2)

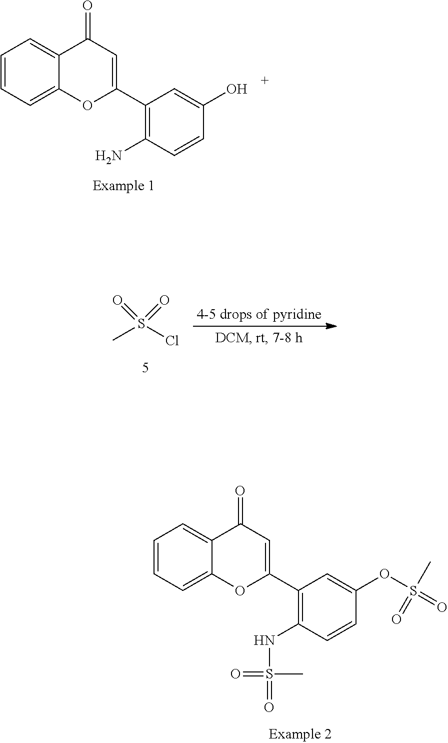

To a solution of Example 1 (0.253 g, 1 mmol) and methanesulfonyl chloride (5, 0.228 g, 2 mmol) in DCM (10 mL), were added 4-5 drops of pyridine and then the reaction mixture was stirred at ambient temperature for 7-8 h. After completion of reaction, ice cold water (50 mL) was added to it and then the reaction mixture was extracted with DCM (2×50 mL). Organic layers were combined, dried over anhydrous Na₂SO₄, filtered and then concentrated under vacuum. The crude was then purified with 50% EtOAc/n-hexane as an eluent to obtain the titled compound (Compound 2).

Yield=70%; Colorless solid; R_{f}=0.3 (50% EtOAc/n-hexane); ¹H NMR (400 MHz, CDCl₃) δ: 9.7 (s, 1H, NH), 8.08-8.06 (m, 1H, ArH), 7.78-7.72 (m, 1H, ArH), 7.65-7.57 (m, 3H, ArH), 7.51-7.42 (m, 2H, ArH), 6.60-6.59 (m, 1H, ArH), 3.04-3.02 (m, 3H, CH₃), 1.28-1.20 (m, 3H, CH₃); MS (ES mass): 410.0 (M+1); HPLC: 97.8%; Column: Cosmicsil Aura ODS 150*4.6 mm, 5 μm; Mobile phase: A) 5 mM NH₄OAc in water, B) ACN; (T/B %): 0/5, 20/90, 25/90, 26/5, 30/5; Flow rate: 1.0 mL/min; Diluent: ACN: Water (80:20); UV: 240.0 nm; Retention time: 11.1 min.

### Example 3: 2-(2-Amino-5-hydroxy-4-iodophenyl)-4H-chromen-4-one (Compound 3)

### Step 1: Preparation of 2-(5-hydroxy-4-iodo-2-nitrophenyl)-4H-chromen-4-one (6)

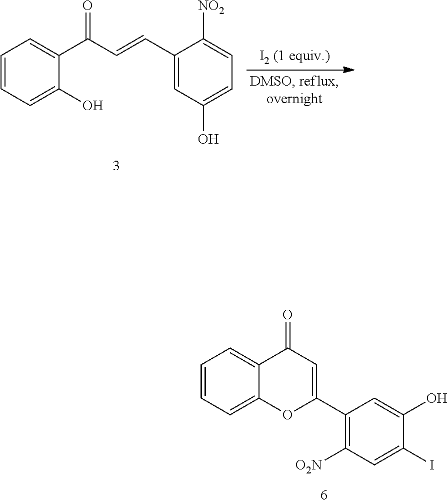 [SMILES: O=C(/C=C/c1cc(O)ccc1[N+](=O)[O-])c1ccccc1O ; O=c1cc(-c2cc(O)c(I)cc2[N+](=O)[O-])oc2ccccc12]

To a solution of intermediate 3 (0.285 g, 1 mmol) in DMSO (10 mL), was added I₂ (0.254 g, 1 mmol) and the resultant mixture was then refluxed at 200° C. for overnight. After completion of reaction, saturated solution of Na₂S₂O₃ (50 mL) was added to it and then the reaction mixture was extracted with EtOAc (3×50 mL). Organic layers were combined, dried over anhydrous Na₂SO₄, filtered and then concentrated under vacuum. The crude product was then purified with 70% EtOAc/n-hexane as an eluent to yield the intermediate 6.
Yield=30%; Brown solid; R_{f}=0.2 (70% EtOAc/n-hexane); ¹H NMR (400 MHz, CDCl₃) δ: 12.27 (broad s, 1H, OH, D₂O exch.), 8.54 (s, 1H, ArH), 8.08-8.06 (m, 1H, ArH), 7.83-7.78 (m, 1H, ArH), 7.57-7.50 (m, 2H, ArH), 6.99 (s, 1H, ArH), 6.61 (s, 1H, ═CH); MS (ES mass): 409.9 (M+1).

Step 2: Preparation of 2-(2-amino-5-hydroxy-4-iodophenyl)-4H-chromen-4-one

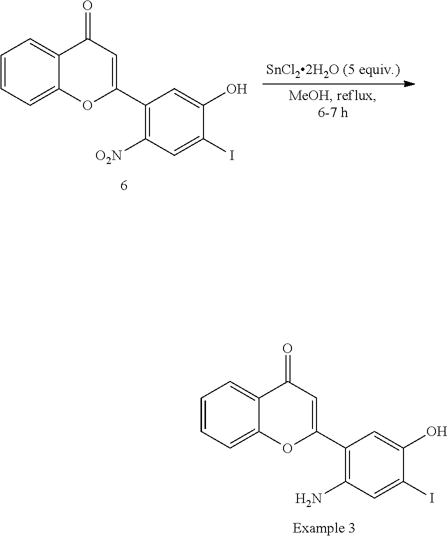 [SMILES: O=c1cc(-c2cc(O)c(I)cc2[N+](=O)[O-])oc2ccccc12 ; Nc1cc(I)c(O)cc1-c1cc(=O)c2ccccc2o1]

This step was carried out according to the method described in step 3 of Example 1. Intermediate 6 (0.379 g, 1 mmol) and SnCl₂.2H₂O (1.13 g, 5 mmol) were refluxed in MeOH (20 mL) for 6-7 h. The crude product was purified by column chromatography (40% EtOAc/n-hexane) to give the titled compound (Compound 3).
Yield=85%; Brick red solid; R_{f}=0.3 (40% EtOAc/n-hexane); ¹H NMR (400 MHz, CDCl₃) δ: 9.62 (s, 1H, OH, D₂O exch.), 8.03 (dd, J=7.9 & 1.5, Hz, 1H, ArH), 7.81-7.77 (m, 1H, ArH), 7.67 (d, J=7.9 Hz, 1H, ArH), 7.48 (t, J=7.2 Hz, 1H, ArH), 7.29 (s, 1H, ArH), 6.92 (s, 1H, ArH), 6.53 (s, 1H, ═CH), 5.37 (broad s, 2H, NH₂); ¹³C NMR (100 MHz, CDCl₃) δ: 171.3 (C═O), 164.1, 156.4, 148.4, 141.0, 134.5, 127.5, 125.9, 125.1, 123.8, 118.9, 117.2, 114.2, 110.2, 91.4 (Cl); MS (ES mass): 379.9 (M+1); HPLC: 89.34%; Column: Eclipse plus C-18 250*4.6 mm, 5 μm; Mobile phase: A) 10 mM K₂HPO₄:KH₂PO₄ in water, B) ACN:H₂O (90:10); (T/B %): 0/5, 20/90, 28/90, 30/5, 35/5; Flow rate: 1.0 mL/min; Diluent: ACN:H₂O (80:20); UV: 210.0 nm; Retention time: 14.4 min.

### Example 4: 2-(2-Amino-5-methoxyphenyl)-4H-chromen-4-one (Compound 4)

### Step 1: Preparation of 2-(5-methoxy-2-nitrophenyl)-4H-chromen-4-one (7)

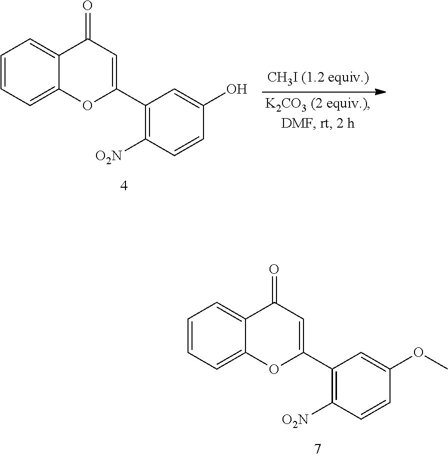 [SMILES: O=c1cc(-c2cc(O)ccc2[N+](=O)[O-])oc2ccccc12 ; COc1ccc([N+](=O)[O-])c(-c2cc(=O)c3ccccc3o2)c1]

To a solution of intermediate 4 (0.283 g, 1 mmol) in DMF (10 mL) were added CH₃I (0.17 g, 1.2 mmol) and K₂CO₃ (0.28 g, 2 mmol). Then the reaction mixture was stirred at room temperature for 2 h. After disappearance of starting material (monitored by TLC), ice cold water (50 mL) was added to it and extracted with EtOAc (2×50 mL). The organic layer was then collected, dried over anhydrous Na₂SO₄, filtered and evaporated. The crude product was purified using column chromatography with 30% EtOAc/n-hexane as an eluent.

Yield=95%; Off-white solid; mp: 183-185° C.; R_{f}=0.45 (30% EtOAc/n-hexane); ¹H NMR (400 MHz, CDCl₃) δ: 8.25 (d, J=9.1 Hz, 1H, ArH), 8.10 (dd, J=7.9 & 1.5, Hz, 1H, ArH), 7.85-7.81 (m, 1H, ArH), 7.58-7.52 (m, 2H, ArH), 7.46 (d, J=2.8 Hz, 1H, ArH), 7.35 (dd, J=9.1 & 2.8, Hz, 1H, ArH), 6.75 (s, 1H, ═CH), 3.96 (s, 3H, CH₃); ¹³C NMR (100 MHz, CDCl₃) δ: 176.8 (C═O), 163.2, 162.4, 155.9, 139.9, 134.7, 129.5, 127.8, 125.9, 124.9, 123.2, 118.3, 117.1, 116.8, 110.9, 56.7 (CH₃); MS (ES mass): 298.0 (M+1); HPLC: 99.6%; Column: XBridge C-18 150*4.6 mm, 5 μm; Mobile phase: A) 0.1% TFA in water, B) ACN; (T/B %): 0/5, 20/90, 30/90, 31/5, 35/5; Flow rate: 1.0 mL/min; Diluent: ACN:Water (80:20); UV: 230.0 nm; Retention time: 14.1 min.

### Step 2: Preparation of 2-(2-amino-5-methoxyphenyl)-4H-chromen-4-one

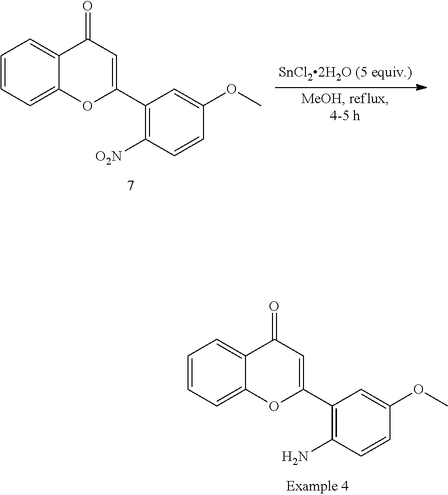 [SMILES: COc1ccc([N+](=O)[O-])c(-c2cc(=O)c3ccccc3o2)c1 ; COc1ccc(N)c(-c2cc(=O)c3ccccc3o2)c1]

This step was carried out according to the method described in step 3 of Example 1. Intermediate 7 (0.297 g, 1 mmol) and SnCl₂.2H₂O (1.13 g, 5 mmol) were refluxed in EtOH (20 mL) for 4-5 h. The crude product was purified by column chromatography (40% EtOAc/n-hexane) to give the titled compound (Compound 4).

Yield=90%; Yellow solid; mp: 118-120° C.; R_{f}=0.38 (40% EtOAc/n-hexane); ¹H NMR (400 MHz, CDCl₃) b: 8.05 (dd, J=7.8 & 1.4, Hz, 1H, ArH), 7.83-7.78 (m, 1H, ArH), 7.73 (d, J=8.2 Hz, 1H, ArH), 7.49 (t, J=7.1 Hz, 1H, ArH), 7.01 (d, J=2.9 Hz, 1H, ArH), 6.92 (dd, J=8.9 & 2.9, Hz, 1H, ArH), 6.82 (d, J=8.9 Hz, 1H, ArH), 6.64 (s, 1H, ═CH), 5.29 (s, 2H, NH₂, D₂O exch.), 3.72 (s, 3H, CH₃); ¹³C NMR (100 MHz, CDCl₃) δ: 176.9 (C═O), 164.5, 155.9, 150.6, 141.4, 133.9, 125.3, 124.6, 123.3, 119.8, 118.7, 118.4, 115.5, 112.8, 109.7, 55.5 (OCH₃); MS (ES mass): 268.1 (M+1); HPLC: 98.6%; Column: Eclipse XDB C-18 150*4.6 mm, 5 μm; Mobile phase: A) 0.05% TFA in water, B) 0.05% TFA in ACN; (T/B %): 0/10, 5/10, 25/90, 30/90, 31/10, 35/10; Flow rate: 1.0 mL/min; Diluent: ACN:Water (80:20); UV: 240.0 nm; Retention time: 13.4 min.

### Example 5: 2-(2-Amino-5-ethoxyphenyl)-4H-chromen-4-one (Compound 5)

### Step 1: Preparation of 2-(5-ethoxy-2-nitrophenyl)-4H-chromen-4-one (8)

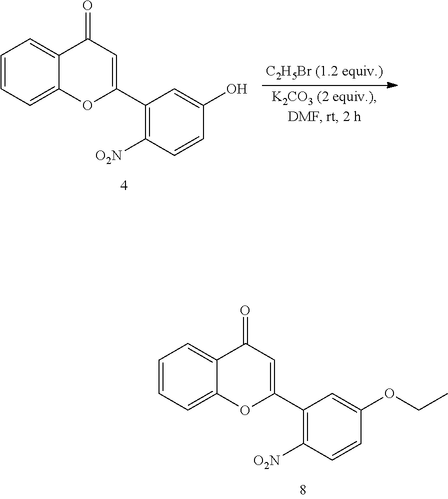 [SMILES: O=c1cc(-c2cc(O)ccc2[N+](=O)[O-])oc2ccccc12 ; CCOc1ccc([N+](=O)[O-])c(-c2cc(=O)c3ccccc3o2)c1]

This step was carried out according to the method depicted in Step 1 of Example 4: A solution of intermediate 4 (0.283 g, 1 mmol), C₂H₅Br (0.13 g, 1.2 mmol) and K₂CO₃ (0.28 g, 2 mmol) in DMF (10 mL) were stirred at rt for 2 h. The crude product was purified by column chromatography (30% EtOAc/n-hexane) to give the desired product (8).

Yield=90%; Off-white solid; mp: 153-155° C.; R_{f}=0.51 (30% EtOAc/n-hexane); ¹H NMR (400 MHz, CDCl₃) δ: 8.23 (d, J=9.1 Hz, 1H, ArH), 8.10 (dd, J=7.9 & 1.5, Hz, 1H, ArH), 7.85-7.81 (m, 1H, ArH), 7.58-7.52 (m, 2H, ArH), 7.43 (d, J=2.8 Hz, 1H, ArH), 7.33 (dd, J=9.1 & 2.8, Hz, 1H, ArH), 6.74 (s, 1H, ═CH), 4.26 (q, J=6.9 Hz, 2H, CH₂), 1.38 (t, J=6.9 Hz, 3H, CH₃); ¹³C NMR (100 MHz, CDCl₃) δ: 176.8 (C═O), 162.5, 163.4, 155.9, 139.8, 134.7, 129.5, 127.8, 125.9, 124.9, 123.2, 118.3, 117.4, 117.1, 110.9, 64.9 (CH₂), 14.29 (CH₃); MS (ES mass): 312.1 (M+1); HPLC: 99.7%; Column: XBridge C-18 150*4.6 mm, 5 μm; Mobile phase: A) 0.1% TFA in water, B) ACN; (T/B %): 0/5, 20/90, 30/90, 31/5, 35/5; Flow rate: 1.0 mL/min; Diluent: ACN:Water (80:10); UV: 230.0 nm; Retention time: 15.2 min.

### Step 2: Preparation of 2-(2-amino-5-ethoxyphenyl)-4H-chromen-4-one

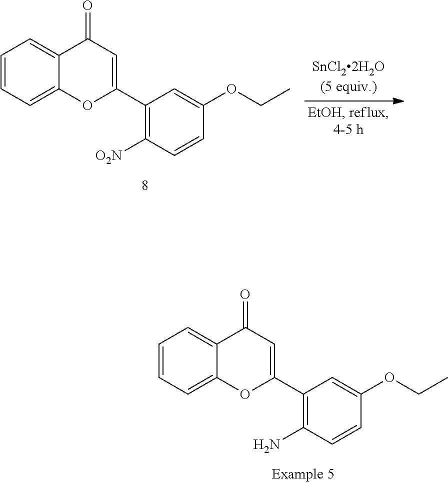 [SMILES: CCOc1ccc([N+](=O)[O-])c(-c2cc(=O)c3ccccc3o2)c1 ; CCOc1ccc(N)c(-c2cc(=O)c3ccccc3o2)c1]

This step was carried out according to the method described in step 3 of Example 1. Intermediate 8 (0.311 g, 1 mmol) and SnCl₂.2H₂O (1.13 g, 5 mmol) were refluxed in EtOH (20 mL) for 4-5 h. The crude product was purified by column chromatography (40% EtOAc/n-hexane) to give the desired product (Compound 5).

Yield=90%; Yellowish brown solid; mp: 88-90° C.; R_{f}=0.4 (40% EtOAc/n-hexane); ¹H NMR (400 MHz, CDCl₃) δ: 8.05 (d, J=6.9 Hz, 1H, ArH), 7.80 (t, J=7.2 Hz, 1H, ArH), 7.73 (d, J=8.3 Hz, 1H, ArH), 7.49 (t, J=7.4 Hz, 1H, ArH), 7.00 (d, J=2.8 Hz, 1H, ArH), 6.91 (dd, J=8.8 & 2.8, Hz, 1H, ArH), 6.80 (d, J=8.9 Hz, 1H, ArH), 6.62 (s, 1H, ═CH), 5.28 (s, 2H, NH₂, D₂O exch.), 3.97 (q, J=6.9 Hz, 2H, CH₂), 1.29 (t, J=6.9 Hz, 3H, CH₃); ¹³C NMR (100 MHz, CDCl₃) δ: 176.9 (C═O), 164.5, 155.9, 149.4, 141.3, 133.9, 125.3, 124.6, 123.3, 120.3, 118.7, 118.4, 115.6, 113.7, 109.7, 63.5 (CH₂), 14.75 (CH₃); MS (ES mass): 282.1 (M+1); HPLC: 95.4%; Column: Eclipse XDB C-18 150*4.6 mm, 5 μm; Mobile phase: A) 0.05% TFA in water, B) 0.05% TFA in ACN; (T/B %): 0/10, 5/10, 25/90, 30/90, 31/10, 35/10; Flow rate: 1.0 mL/min; Diluent: ACN:Water (80:20); UV: 240.0 nm; Retention time: 14.7 min.

### Example 6: 2-(2-Amino-5-(prop-2-ynyloxy)phenyl)-4H-chromen-4-one (Compound 6)

### Step 1: Preparation of 2-(2-nitro-5-(prop-2-ynyloxy)phenyl)-4H-chromen-4-one (9)

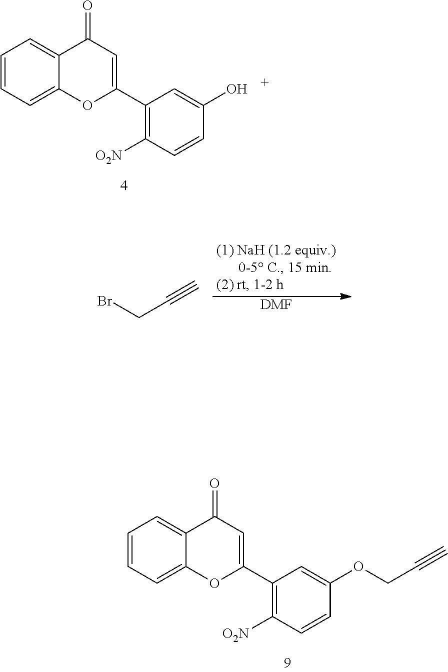

To a solution of intermediate 4 (0.283 g, 1 mmol) in DMF (10 mL) was added NaH (0.024 g, 1 mmol) at about 0-5° C. and the reaction mixture was then stirred for 15 min. After that propargyl bromide (0.143 g, 1.2 mmol) was added to the reaction mixture and then continued stirring at room temperature for 1-2 h. After disappearance of starting material (monitored by TLC), ice cold water (50 mL) was added to it and reaction mixture was extracted with EtOAc (2×50 mL). The organic layer was then dried over Na₂SO₄, filtered, and evaporated. The crude product was purified using column chromatography with 30% EtOAc/n-hexane as an eluent to give the desired product 9.

Yield=90%; Yellow solid; R_{f}=0.48 (30% EtOAc/n-hexane); ¹H NMR (400 MHz, CDCl₃) δ: 8.28 (d, J=9.1 Hz, 1H, ArH), 8.10 (dd, J=7.9 & 1.5 Hz, 1H, ArH), 7.86-7.81 (m, 1H, ArH), 7.58-7.52 (m, 3H, ArH), 7.42 (dd, J=9.1 & 2.8 Hz, 1H, ArH), 6.75 (s, 1H, ═CH), 5.07 (d, J=2.4 Hz, 2H, CH₂), 3.73 (t, J=2.4 Hz, 1H, ≡CH); ¹³C NMR (100 MHz, CDCl₃) δ: 176.8 (C═O), 162.1, 160.9, 155.8, 140.6, 134.8, 129.2, 127.7, 125.9, 124.9, 123.2, 118.3, 118.0, 117.5, 110.9, 79.5 (C≡CH), 77.9 (C≡CH), 56.7 (CH₂); MS (ES mass): 322.0 (M+1).

### Step 2: Preparation of 2-(2-amino-5-(prop-2-ynyloxy)phenyl)-4H-chromen-4-one

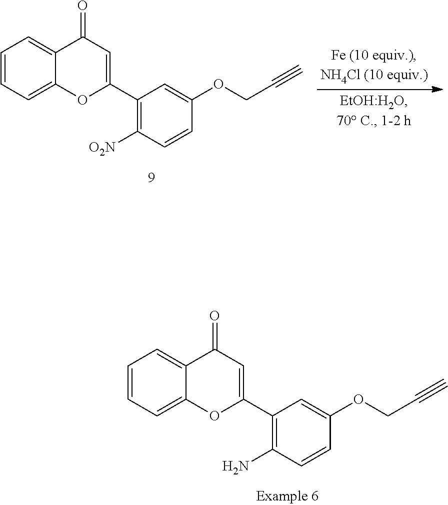 [SMILES: C#CCOc1ccc([N+](=O)[O-])c(-c2cc(=O)c3ccccc3o2)c1 ; C#CCOc1ccc(N)c(-c2cc(=O)c3ccccc3o2)c1]

To a solution of intermediate 9 (0.321 g, 1 mmol) in 4:1 EtOH/H₂O (20 mL), were added Fe powder (0.56 g, 10 mmol) and NH₄Cl (0.535 g, 10 mmol). The reaction mixture was then stirred at 70° C. for 1-2 h. The mixture was filtered through a pad of celite and the pad was washed with EtOH (3×30 mL). The filtrate was concentrated, and the resulting material was suspended in H₂O (50 mL) and extracted with EtOAc (3×100 mL). The combined organics were washed with brine (50 mL), dried (over anhydrous Na₂SO₄), and concentrated. The resulting residue was purified by silica gel column chromatography (30% EtOAc/n-hexane) to give the compound 6.

Yield=90%; Yellow solid; mp: 153-155° C.; R_{f}=0.35 (30% EtOAc/n-hexane); ¹H NMR (400 MHz, CDCl₃) δ: 8.06 (dd, J=7.9 & 1.6, Hz, 1H, ArH), 7.83-7.79 (m, 1H, ArH), 7.72 (d, J=7.8 Hz, 1H, ArH), 7.52-7.48 (m, 1H, ArH), 7.12 (d, J=2.9 Hz, 1H, ArH), 6.97 (dd, J=8.9 & 2.9 Hz, 1H, ArH), 6.82 (d, J=8.9 Hz, 1H, ArH), 6.62 (s, 1H, ═CH), 5.38 (s, 2H, NH₂, D₂O Exch.), 4.74 (d, J=2.4 Hz, 2H, CH₂), 3.55 (t, J=2.3 Hz, 1H, ≡CH); ¹³C NMR (100 MHz, CDCl₃) δ: 177.4 (C═O), 164.9, 156.4, 148.8, 142.6, 134.4, 125.8, 125.1, 123.8, 121.4, 119.2, 118.7, 115.8, 115.2, 110.1, 80.1 (C≡CH), 78.5 (C≡CH), 56.7 (CH₂); MS (ES mass): 292.1 (M+1); HPLC: 98.8%; Column: Cosmicsil C18 150*4.6 mm, 5 μm; Mobile phase: A) 0.1% TFA in water, B) ACN; (T/B %): 0/30, 20/95, 30/95, 31/30, 35/30; Flow rate: 1.0 mL/min; Diluent: ACN:Water (80:20); UV: 240.0 nm; Retention time: 6.6 min.

### Example 7: N-(2-(4-Oxo-4H-chromen-2-yl)-4-(prop-2-ynyloxy)phenyl)benzene sulfonamide (Compound 7)

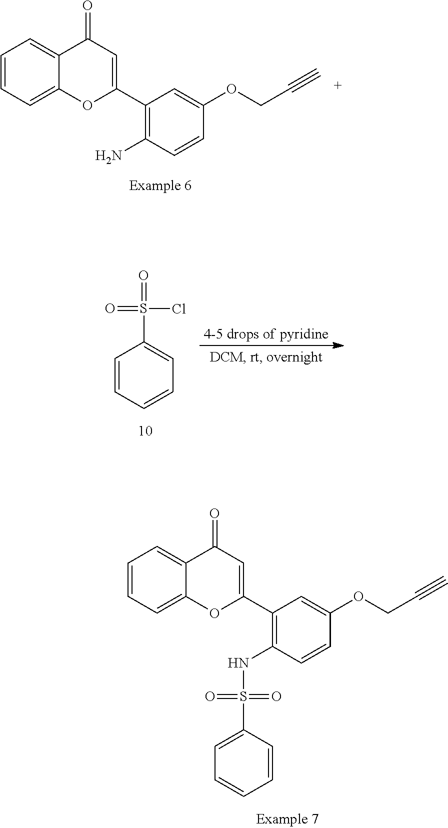 [SMILES: C#CCOc1ccc(N)c(-c2cc(=O)c3ccccc3o2)c1 ; O=S(=O)(Cl)c1ccccc1 ; C#CCOc1ccc(NS(=O)(=O)c2ccccc2)c(-c2cc(=O)c3ccccc3o2)c1]

This step was carried out according to the method described in Example 2. Compound 6 (0.291 g, 1 mmol) and benzenesulfonyl chloride (10, 0.21 g, 1.2 mmol) were stirred in DCM (20 mL) at rt for overnight. The crude product was purified by column chromatography (30% EtOAc/n-hexane) to give the desired compound 7.

Yield=80%; Pale yellow solid; mp: 152-154° C.; R_{f}=0.4 (30% EtOAc/n-hexane); ¹H NMR (400 MHz, CDCl₃) δ: 9.91 (s, 1H, NH, D₂O exch.), 8.03 (dd, J=7.9 & 1.6 Hz, 1H, ArH), 7.86-7.81 (m, 1H, ArH), 7.63 (d, J=7.9 Hz, 1H, ArH), 7.53-7.49 (m, 3H, ArH), 7.45-7.41 (m, 1H, ArH), 7.35-7.31 (m, 2H, ArH), 7.27-7.26 (m, 1H, ArH), 7.13-7.12 (m, 2H, ArH), 6.93 (s, 1H, ═CH), 4.87 (d, J=2.4 Hz, 2H, CH₂), 3.64 (t, J=2.3 Hz, 1H, ≡CH); ¹³C NMR (100 MHz, CDCl₃) δ: 176.7 (C═O), 162.2, 156.1, 155.7, 140.1, 134.1, 132.5, 131.1, 130.1, 128.9 (2C), 126.9, 126.3 (2C), 125.3, 124.6, 123.2, 118.7, 118.0, 115.9, 111.7, 78.7 (C≡CH), 78.7 (C≡CH), 55.9 (CH₂); MS (ES mass): 432.0 (M+1); HPLC: 99.5%, Column: EClips XDB C18 150*4.6 mm, 5 μm; Mobile phase: A) 0.05% TFA in water, B) 0.05% TFA in ACN:Water; (T/B %): 0/30, 20/95, 27/95, 31/30, 35/30; Flow rate: 1.0 mL/min; Diluent: ACN:Water (50:50); UV: 235.0 nm; Retention time: 11.4 min.

### Example 8: N-(4-Hydroxy-2-(4-oxo-4H-chromen-2-yl)phenyl)benzene Sulfonamide (Compound 8)

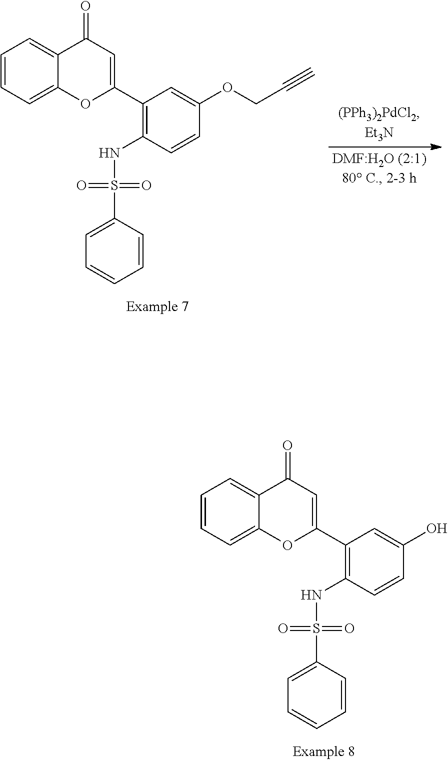 [SMILES: C#CCOc1ccc(NS(=O)(=O)c2ccccc2)c(-c2cc(=O)c3ccccc3o2)c1 ; O=c1cc(-c2cc(O)ccc2NS(=O)(=O)c2ccccc2)oc2ccccc12]

A mixture of compound 7 (0.43 g, 1 mmol), bis-(triphenylphosphine)palladium (II) dichloride (0.028 g, 0.04 mmol) and triethylamine (0.81 g, 8 mmol) in 2:1 DMF-H₂O (10 mL) was heated to 80° C. under inert atmosphere for 2-3 h with stirring. After completion of the reaction, the mixture was poured into water (30 mL) with stirring, acidified with 3N HCl and then extracted with ethyl acetate (3×150 mL). Organic layers were collected, combined, washed with water (2×100 mL), dried over anhydrous Na₂SO₄, filtered and concentrated under vacuum. The residue was purified by column chromatography (30% EtOAc/n-hexane) to afford the desired compound 8.

Yield=65%; Off-white solid; mp: >200° C.; R_{f}=0.2 (30% EtOAc/n-hexane); ¹H NMR (400 MHz, CDCl₃) δ: 9.99 (s, 1H, NH, D₂O exch.), 9.73 (s, 1H, OH, D₂O exch.), 8.03-8.01 (m, 1H, ArH), 7.84-7.79 (m, 1H, ArH), 7.63 (d, J=8.2 Hz, 1H, ArH), 7.52-7.47 (m, 3H, ArH), 7.41-7.37 (m, 1H, ArH), 7.32-7.28 (m, 2H, ArH), 7.01-6.96 (m, 2H, ArH), 6.88 (dd, J=8.7 & 2.8 Hz, 1H, ArH), 6.35 (s, 1H, ═CH); ¹³C NMR (100 MHz, CDCl₃) δ: 176.7 (C═O), 162.4, 156.5, 156.1, 140.1, 134.0, 132.4, 131.7, 130.9, 128.8 (2C), 126.4 (2C), 125.3, 124.6, 124.6, 123.2, 118.6, 118.3, 116.1, 111.5; MS (ES mass): 394.0 (M+1); HPLC: 99.5%; Column: X-Bridge C-18 150*4.6 mm, 5 μm; Mobile phase: A) 5 mM NH₄OAc in water, B) ACN; (T/B %): 0/5, 20/90, 30/90, 31/5, 35/5; Flow rate: 1.0 mL/min; Diluent: ACN:H₂O (80:20); UV: 210.0 nm; Retention time: 11.25 min.

### Example 9: 4-Methyl-N-(2-(4-oxo-4H-chromen-2-yl)-4-(prop-2-ynyloxy)phenyl) benzenesulfonamide (Compound 9)

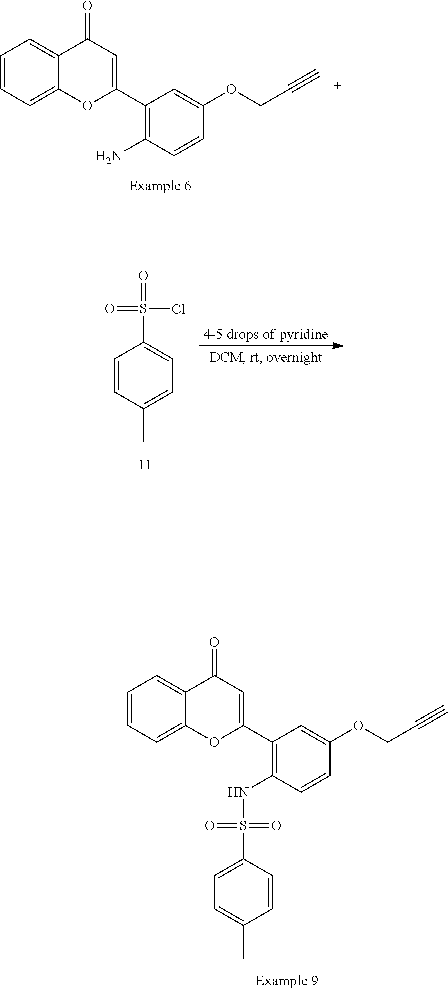 [SMILES: C#CCOc1ccc(N)c(-c2cc(=O)c3ccccc3o2)c1 ; Cc1ccc(S(=O)(=O)Cl)cc1 ; C#CCOc1ccc(NS(=O)(=O)c2ccc(C)cc2)c(-c2cc(=O)c3ccccc3o2)c1]

This step was carried out according to the method described in Example 2. Compound 6 (0.291 g, 1 mmol) and p-toluene sulfonyl chloride (11, 0.23 g, 1.2 mmol) were stirred in DCM (20 mL) at rt for overnight. The crude product was purified by column chromatography (20% EtOAc/n-hexane) to give the desired compound 9.

Yield=80%; Colorless solid; mp: 198-200° C.; R_{f}=0.38 (20% EtOAc/n-hexane); ¹H NMR (400 MHz, CDCl₃) δ: 9.80 (s, 1H, NH, D₂O exch.), 8.04-8.01 (m, 1H, ArH), 7.86-7.81 (m, 1H, ArH), 7.65 (d, J=8.2 Hz, 1H, ArH), 7.53-7.49 (m, 1H, ArH), 7.33 (d, J=8.2 Hz, 2H, ArH), 7.24-7.14 (m, 3H, ArH), 7.03 (d, J=8.0 Hz, 2H, ArH), 6.36 (s, 1H, ═CH), 4.88 (d, J=2.2 Hz, 2H, CH₂), 3.62-3.61 (m, 1H, —CH), 2.15 (s, 3H, CH₃); ¹³C NMR (100 MHz, CDCl₃) δ: 176.6 (C═O), 161.9, 156.0, 155.8, 120.9, 137.2, 134.1, 131.3, 131.0, 129.4 (2C), 126.9, 126.3 (2C), 125.4, 124.5, 123.2, 118.7, 118.0, 115.9, 111.6, 78.8 (C≡CH), 78.7 (C≡CH), 55.9 (OCH₂), 20.8 (CH₃); MS (ES mass): 446.1 (M+1); HPLC: 99.7%, Column: X Bridge C18 150*4.6 mm, 5 μm; Mobile phase: A) 5 mM NH₄OAc in water B) ACN; (T/B %): 0/10, 3/10, 15/95, 23/95, 25/10, 30/10; Flow rate: 1.0 mL/min; Diluent: ACN: Water (80:20); UV: 235.0 nm; Retention time: 12.79 min.

### Example 10: N-(4-Hydroxy-2-(4-oxo-4H-chromen-2-yl)phenyl)-4-methyl Benzenesulfonamide (Compound 10)

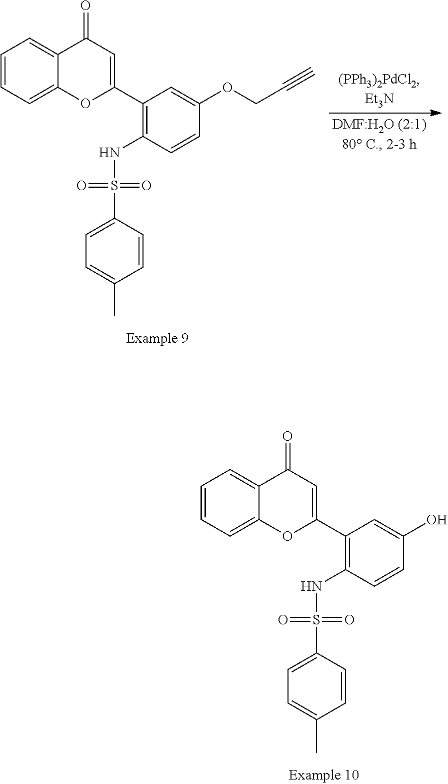 [SMILES: C#CCOc1ccc(NS(=O)(=O)c2ccc(C)cc2)c(-c2cc(=O)c3ccccc3o2)c1 ; Cc1ccc(S(=O)(=O)Nc2ccc(O)cc2-c2cc(=O)c3ccccc3o2)cc1]

This step was carried out according to the method described in Example 8. A mixture of compound 9 (0.45 g, 1 mmol), bis-(triphenylphosphine)palladium (II) dichloride (0.028 g, 0.04 mmol) and triethylamine (0.81 g, 8 mmol) in 2:1 DMF-H₂O (10 mL) was heated to 80° C. under inert atmosphere for 2-3 h with stirring. The crude product was purified by column chromatography (30% EtOAc/n-hexane) to give the desired compound.

Yield=65%; Off-white solid; mp: >200° C.; R_{f}=0.2 (30% EtOAc/n-hexane); ¹H NMR (400 MHz, CDCl₃) δ: 9.99 (s, 1H, NH, D₂O exch.), 9.63 (s, 1H, OH, D₂O exch.), 8.03-8.0 (m, 1H, ArH), 7.84-7.8 (m, 1H, ArH), 7.63 (d, J=8.2 Hz, 1H, ArH), 7.52-7.48 (m, 1H, ArH), 7.29 (d, J=8.2 Hz, 2H, ArH), 7.10-7.97 (m, 4H, ArH), 6.91 (dd, J=8.7 & 2.9 Hz, 1H, ArH), 6.32 (s, 1H, ═CH), 2.1 (s, 3H, CH₃); ¹³C NMR (100 MHz, CDCl₃) δ: 176.6 (C═O), 162.2, 156.5, 155.9, 142.7, 137.3, 134.0, 131.8, 131.8, 129.3 (2C), 126.3 (2C), 125.3, 124.5, 124.5, 123.1, 118.5, 118.3, 115.9, 111.4, 20.8 (CH₃); MS (ES mass): 408.1 (M+1); HPLC: 98.72%; Column: X-Bridge C-18 150*4.6 mm, 5 μm; Mobile phase: A) 5 mM NH₄OAc in water, B) ACN; (T/B %): 0/5, 20/90, 30/90, 31/5, 35/5; Flow rate: 1.0 mL/min; Diluent: ACN:H₂O (80:20); UV: 235.0 nm; Retention time: 11.99 min.

### Example 11: N-(2-(4-Oxo-4H-chromen-2-yl)-4-(prop-2-ynyloxy)phenyl) benzamide (Compound 11)

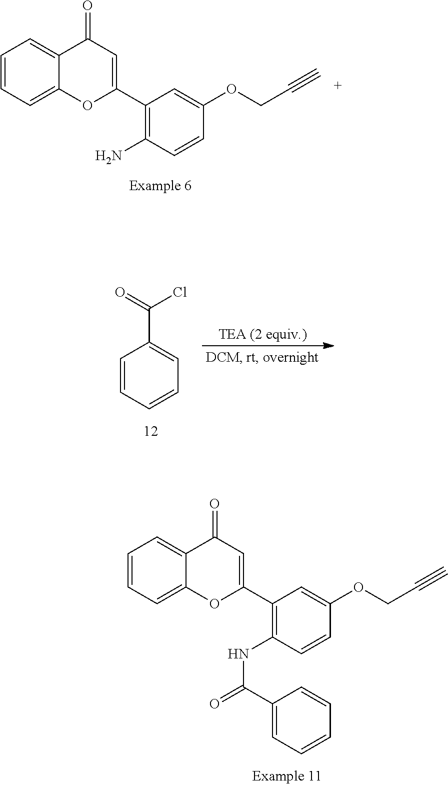 [SMILES: C#CCOc1ccc(N)c(-c2cc(=O)c3ccccc3o2)c1 ; O=C(Cl)c1ccccc1 ; C#CCOc1ccc(NC(=O)c2ccccc2)c(-c2cc(=O)c3ccccc3o2)c1]

To a solution of Compound 6 (0.291 g, 1 mmol) in DCM (10 mL) were added triethylamine (0.202 g, 2 mmol) and benzoyl chloride (12, 0.168 g, 1.2 mmol). Then the reaction mixture was stirred for overnight at ambient temperature. After completion of the reaction, ice cold water (50 mL) was added to it and then the reaction mixture was extracted with DCM (2×50 mL). Organic layers were combined, dried over anhydrous Na₂SO₄, filtered and then concentrated under vacuum. The crude product was then purified by column chromatography using 20% EtOAc/n-hexane as an eluent to give the desired product (Compound 11).

Yield=85%; Colorless solid; mp: 154-156° C.; R_{f}=0.3 (20% EtOAc/n-hexane); ¹H NMR (400 MHz, CDCl₃) δ: 10.24 (s, 1H, NH, D₂O exch.), 8.03-8.01 (m, 1H, ArH), 7.87-7.85 (m, 2H, ArH), 7.76-7.72 (m, 1H, ArH), 7.57-7.42 (m, 7H, ArH), 7.26 (dd, J=8.8 & 2.9 Hz, 1H, ArH), 6.64 (s, 1H, ═CH), 4.94 (d, J=2.3 Hz, 2H, OCH₂), 3.63 (t, J=2.3 Hz, 1H, ≡CH); ¹³C NMR (100 MHz, CDCl₃) δ: 176.9 (C═O), 165.7 (HN—C═O), 163.5, 155.9, 154.9, 134.2, 134.1, 131.6, 129.3, 129.1, 129.0 (2C), 128.4 (2C), 127.5, 125.4, 124.8, 123.2, 118.2, 118.1, 115.3, 110.3, 78.9 (C≡CH), 78.6 (C≡CH), 55.9 (CH₂); MS (ES mass): 396.1 (M+1); HPLC: 98.6%; Column: X-Bridge C18 150*4.6 mm, 5 μm; Mobile phase: A) 5 mM NH₄OAc in water, B) ACN; (T/B %): 0/10, 23/90, 30/90, 31/90, 35/10; Flow rate: 1.0 mL/min; Diluent: ACN:H₂O (80:20); UV: 240.0 nm; Retention time: 14.31 min.

### Example 12: N-(4-Hydroxy-2-(4-oxo-4H-chromen-2-yl)phenyl)benzamide (Compound 12)

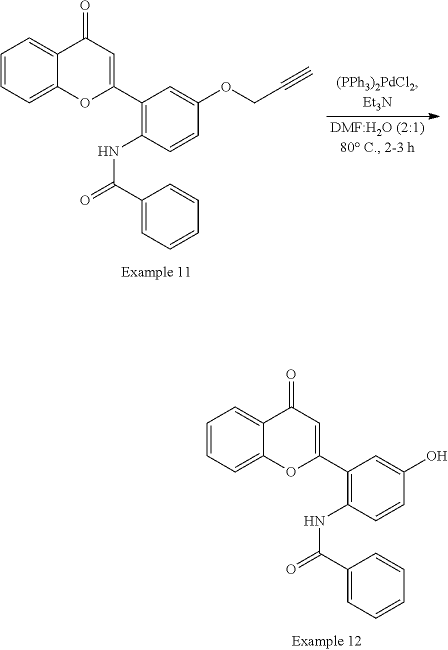 [SMILES: C#CCOc1ccc(NC(=O)c2ccccc2)c(-c2cc(=O)c3ccccc3o2)c1 ; O=C(Nc1ccc(O)cc1-c1cc(=O)c2ccccc2o1)c1ccccc1]

This step was carried out according to the method described in Example 8. A mixture of Compound 11 (0.395 g, 1 mmol), bis-(triphenylphosphine)palladium (II) dichloride (0.028 g, 0.04 mmol) and triethylamine (0.81 g, 8 mmol) in 2:1 DMF-H₂O (10 mL) was heated to 80° C. under inert atmosphere for 2-3 h with stirring. The crude product was purified by column chromatography (30% EtOAc/n-hexane) to give the desired Compound 12.

Yield=60%; Colorless solid; mp: >200° C.; R_{f}=0.22 (30% EtOAc/n-hexane); ¹H NMR (400 MHz, CDCl₃) δ: 10.11 (s, 1H, NH, D₂O exch.), 9.89 (s, 1H, OH, D₂O exch.), 8.02-7.99 (m, 1H, ArH), 7.86-7.84 (m, 2H, ArH), 7.77-7.73 (m, 1H, ArH), 7.56-7.50 (m, 2H, ArH), 7.48-7.44 (m, 3H, ArH), 7.38-7.36 (m, 1H, ArH), 7.17 (d, J=2.3 Hz, 1H, ArH), 7.02 (dd, J=8.6 & 2.8 Hz, 1H, ArH), 6.53 (s, 1H, ═CH); ¹³C NMR (100 MHz, CDCl₃) δ: 176.9 (C═O), 165.7 (HN—C═O), 163.6, 155.9, 155.5, 134.3, 134.1, 131.5, 129.5, 129.4, 128.3 (2C), 127.4 (2C), 127.0, 125.4, 124.7, 123.2, 118.2 (2C), 115.3, 110.1; MS (ES mass): 358.1 (M+1); HPLC: 98.46%; Column: X-Bridge C-18 150*4.6 mm, 5 μm; Mobile phase: A) 5 mM NH₄OAc in water, B) ACN; (T/B %): 0/5, 20/90, 25/90, 26/5, 30/5; Flow rate: 1.0 mL/min; Diluent: ACN:H₂O (80:20); UV: 240.0 nm; Retention time: 10.83 min.

The compounds appearing in the following Table 1 were prepared by analogy to the procedure described above.

TABLE 1

| Compound No. | Structure | Characterization data |
|---|---|---|
| 13 | 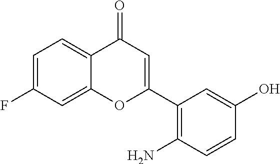 [SMILES: Nc1ccc(O)cc1-c1cc(=O)c2ccc(F)cc2o1] | Yield = 90%; Yellow colored solid; mp: 200-202° C.; R_{f} = 0.3 (40% EtOAcIn- hexane); ¹H NMR (400 MHz, CDCl₃) δ: 8.81 (s, 1H, OH, D₂O exch.), 8.10 (dd, J = 8.8 & 6.5 Hz, 1H), 7.67 (dd, J = 9.7 & 2.4 Hz, 1H), 7.36 (dt, J = 8.7 & 2.4 Hz, 1H), 6.85 (d, J = 2.5 Hz, 1H), 6.78-6.71 (m, 2H), 6.54 (s, 1H, ═CH), 5.10 (broad s, 2H, NH₂, D₂O exch); ¹³C NMR (100 MHz, CDCl₃) δ: 176.0 (C═O), 166.1 (d, J = 251.1 Hz, CF), 164.9, 163.6, 157.0 (d, J = 14.2), 148.3, 139.1, 127.5 (d, J = 10.9 Hz), 120.5 (d, J = 2.2 Hz), 118.5, 115.7, 114.2, 113.9 (d, J = 23.1 Hz), 109.5, 105.5 (d, J = 25.6 Hz); MS (ES mass): 272.0 (M + 1); HPLC: 97.1%; Column: X- Bridge C18 150*4.6 mm, 5 μm; Mobile phase: A) 0.1% TFA in water B) ACN; (T/B %): 0/10, 20/90, 30/90, 31/10, 35/10; Flow rate: 1.0 mL/min; Diluent: ACN:H₂O (80:20); UV: 235.0 nm; Retention time: 6.0 min. |
| 14 | 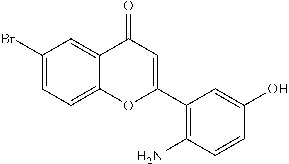 [SMILES: Nc1ccc(O)cc1-c1cc(=O)c2cc(Br)ccc2o1] | Yield = 90%; Beige solid; mp: >200° C.; R_{f} = 0.38 (40% EtOAcIn-hexane); ¹H NMR (400 MHz, CDCl₃) δ: 8.82 (s, 1H, OH, D₂O exch.), 8.11 (d, J = 2.5 Hz, 1H), 7.96 (dd, J = 8.9 & 2.5 Hz, 1H), 7.70 (d, J = 8.9 Hz, 1H), 6.86 (d, J = 2.6 Hz, 1H), 6.8-6.7 (m, 2H), 6.59 (s, 1H, ═CH), 5.1 (broad s, 2H, NH₂, D₂O exch.); ¹³C NMR (100 MHz, CDCl₃) δ: 175.6 (C═O), 164.9, 154.9, 148.3, 140.1, 136.5, 126.7, 124.9, 121.3, 120.6, 118.6, 117.7, 115.7, 114.2, 109.5; MS (ES mass): 333.9 (M + 2); HPLC: 95.1%, Column: X-Bridge C18, 150*4.6 mm, 5 μm; Mobile phase: A) 5 mM NH₄OAc in water B) ACN; (T/B %): 0/20, 20/90, 30/90, 31/20, 35/20; Flow rate: 1.0 mL/min; UV: 245.0 nm; Retention time: 7.9 min. |
| 15 | 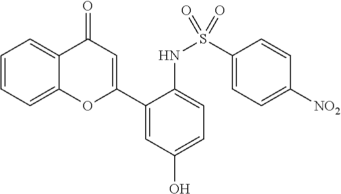 [SMILES: O=c1cc(-c2cc(O)ccc2NS(=O)(=O)c2ccc([N+](=O)[O-])cc2)oc2ccccc12] | Yield = 85%; Beige solid; mp: >200° C.; R_{f} = 0.32 (40% EtOAcIn-hexane); ¹H NMR (400 MHz, CDCl₃) δ: 10.17 (broad s, 1H, NH, D₂O exch.), 10.12 (s, 1H, OH, D₂O exch.), 8.00 (d, J = 8.9 Hz, 2H), 7.90 (dd, J = 7.9 & 1.5 Hz, 1H), 7.80-7.76 (m, 1H), 7.79 (d, J = 8.9 Hz, 2H), 7.60 (d, J = 8.2 Hz, 1H), 7.50-7.44 (m, 1H), 7.10 (d, J = 8.6 Hz, 1H), 6.99 (d, J = 2.8, 1H), 6.95- 6.93 (m, 1H), 6.35 (s, 1H, ═CH); ¹³C NMR (100 MHz, CDCl₃) δ: 176.4 (C═O), 162.0, 157.1, 155.8, 148.9, 146.0, 1342, 132.6, 132.3, 127.8 (2C), 127.2, 125.5, 124.5, 124.1 (2C), 122.9, 118.5, 118.4, 116.2, 111.5; MS (ES mass): 439.0 (M + 1); HPLC: 98.9%, Column: X-Bridge C18, 150*4.6 mm, 5 μm; Mobile phase: A) 5 mM NH₄OAc in water B) ACN; (T/B %): 0/20, 20/90, 30/90, 31/20, 35/20; Flow rate: 1.0 mL/min; Diluent: ACN:H₂O (80:20); UV: 220.0 nm; Retention time: 10.8 min. |
| 16 | 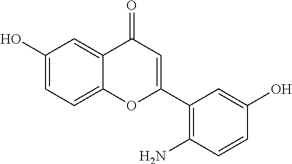 [SMILES: Nc1ccc(O)cc1-c1cc(=O)c2cc(O)ccc2o1] | Yield = 80%; Reddish powder; mp: 130- 132° C.; R_{f} = 0.27 (50% EtOAcIn- hexane); ¹H NMR (400 MHz, CDCl₃) δ: 9.95 (s, 1H, OH, D₂O exch.), 8.79 (s, 1H, OH, D₂O exch.), 7.57 (d, J = 8.9 Hz, 1H), 7.32 (d, J = 2.9 Hz, 1H), 7.22 (dd, J = 8.9 & 3.0 Hz, 1H), 6.84 (d, J = 2.4 Hz, 1H), 6.76-6.70 (m, 2H), 6.47 (s, 1H, ═CH), 5.04 (broad s, 2H, NH₂, D₂O exch.); ¹³C NMR (100 MHz, CDCl₃) δ: 176.7 (C═O), 164.0, 154.7, 149.6, 148.4, 139.8, 124.2, 122.7, 120.1, 119.8, 118.5, 116.3, 114.2, 108.5, 107.4; MS (ES mass): 270.0 (M + 1); HPLC: 92.1%, Column: X-Bridge Phenyle, 150*4.6 mm, 3.5 μm; Mobile phase: A) 0.05% TFA in water B) ACN:H₂O (90:10); (T/B %): 0/2, 5/2, 20/90, 25/90, 26/2, 30/2; Flow rate: 1.0 mL/min; Diluent: ACN:H₂O (10:90); UV: 210.0 nm; Retention time: 11.0 min. |
| 17 | 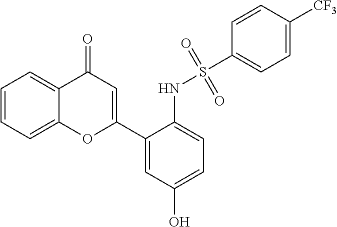 [SMILES: O=c1cc(-c2cc(O)ccc2NS(=O)(=O)c2ccc(C(F)(F)F)cc2)oc2ccccc12] | Yield = 87%; Yellow powder; mp: 160-162° C.; R_{f} = 0.32 (40% EtOAcIn-hexane); ¹H NMR (400 MHz, CDCl₃) δ: 10.07 (broad s, 2H, NH & OH, D₂O exch.), 7.98 (dd, J = 7.9 & 1.5 Hz, 1H), 7.83-7.78 (m, 1H), 7.68-7.59 (m, 5H), 7.51-7.47 (m, 1H), 7.06-7.02 (m, 2H), 6.90 (dd, J = 8.7 & 2.9 Hz, 1H), 6.37 (s, 1H, ═CH); ¹³C NMR (100 MHz, CDCl₃) δ: 176.5 (C═O), 162.1, 156.8, 155.9, 147.7, 144.3, 144.2, 139.9, 134.1, 131.9, 131.8, 127.2, 126.0 (q, J = 3.7 Hz, CF₃), 125.4, 124.6, 122.9, 118.44, 118.42, 116.2, 111.4; MS (ES mass): 461.9 (M + 1); HPLC: 97.8%, Column: X- Bridge C18, 150*4.6 mm, 5 μm; Mobile phase: A) 5 mM NH₄OAc in water B) ACN; (T/B %): 0/10, 23/90, 30/90, 31/10, 35/10; Flow rate: 1.0 mL/min; Diluent: ACN:H₂O (80:20); UV: 215.0 nm; Retention time: 13.9 min. |
| 18 | 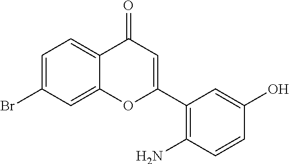 [SMILES: Nc1ccc(O)cc1-c1cc(=O)c2ccc(Br)cc2o1] | Yield = 85%; Peru colored solid; mp: >200° C.; R_{f} = 0.37 (40% EtOAcIn-hexane); ¹H NMR (400 MHz, CDCl₃) δ: 8.81 (s, 1H, OH, D₂O exch.), 8.10 (d, J = 1.8 Hz, 1H), 7.95 (d, J = 8.5 Hz, 1H), 7.66 (dd, J = 8.5 & 1.8 Hz, 1H), 6.85 (d, J = 2.6 Hz, 1H), 6.78-6.71 (m, 2H), 6.55 (s, 1H, ═CH), 5.21 (broad s, 2H, NH₂, D₂O exch.); ¹³C NMR (100 MHz, CDCl₃) δ: 176.2 (C═O), 164.9, 156.1, 148.3, 140.1, 128.5, 126.8, 126.5, 122.4, 121.5, 120.6, 118.6, 115.6, 114.3, 109.6; MS (ES mass): 333.8 (M + 1); HPLC: 85.9%, Column: X-Bridge C-18, 150*4.6 mm, 5 μm; Mobile phase: A) 0.05% TFA in water B) 0.05% TFA in ACN; (T/B %): 0/5, 25/90, 35/90, 36/5, 40/5; Flow rate: 1.0 mL/min; Diluent: ACN:H₂O (80:20); UV: 210.0 nm; Retention time: 9.7 min. |

### Pharmacological Assay

### Cloning and Expression of 12R-LOX:

Human 12R-LOX gene was cloned and expressed as an N-terminally His-tagged fusion protein. Coding region of the cDNA was amplified from the basic vector pCMV6Neo-12R-LOX (OriGene, USA) and was ligated into pET28(b), a prokaryotic protein expression vector. E. coli BL21 Rossetta DE3 (an engineered strain of BL21) was transformed with the pET28(b) recombinant vector containing Kanamycin and chloramphenicol resistance gene as the selection markers. Single colony selected on kanamycin and chloramphenicol was checked for the expression of the protein. For this single colony was cultured at 37° C. in LB medium containing 50 mg/mL kanamycin and 25 mg/mL chloramphenicol to reach an absorbance of 0.6 at 600 nm. The cultured bacteria were then induced to express recombinant protein by using isopropyl β-D-thiogalactopyranoside (IPTG) (1 mM final concentration) at 18° C. The culture was incubated for 8-12 h and then the culture was spun down to collect the bacterial cells. The cells were re-suspended in the required amount of phosphate buffer saline (PBS) containing protease inhibitor. The cells were then lysed by sonication to extract the protein. The cells were sonicated for 5 min. and then debris was removed by centrifugation at 13000 rpm for 30 min at 4° C. The clear supernatant was collected and was used as the source of enzyme.

### Purification of the Recombinant 12R-LOX Protein:

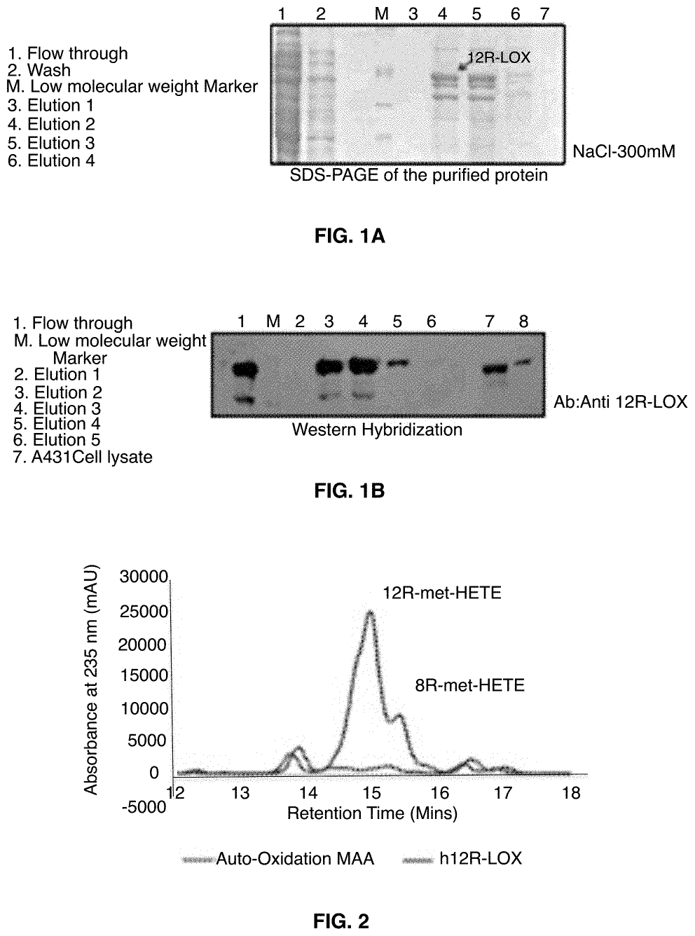

Purification of the recombinant 12R-LOX protein was performed using nickel-nitrilotriacetic acid (Ni-NTA) metal-affinity chromatography as per manufacturer's protocol. Briefly, Ni-NTA slurry (500 microliters for 150 mL of culture) was added to the cleared bacterial lysate and mixed gently by shaking at 4° C. for 60 min. Then the lysate—Ni-NTA slurry was loaded into a column. The column was then washed with 10 mL of washing buffer (50 mM phosphate buffer with 300 mM sodium chloride) containing 20 mM imidazole to remove loosely bound proteins. Then tightly bound proteins were eluted from the column with elution buffer (50 mM phosphate buffer with 300 mM sodium chloride) containing 300 mM imidazole. The purified protein fractions were then analyzed further by SDS-PAGE and western blotting to confirm the presence of 12R-LOX (FIGS. 1a and 1b).

### Standardization of the Activity Assay:

Fractions with human 12R-LOX protein were added to a total volume of 0.3 mL of phosphate buffered saline (pH 7.4) and incubated with substrate, arachidonic acid methyl ester. The mixture was vortexed and incubated for 15 min at 37° C. After the incubation time, hydroperoxy fatty acids formed in the reaction were reduced to their more stable hydroxy derivatives by addition of sodium borohydride. After 5 min the reaction was acidified to pH 3 with 50 μL of acetic acid and then the proteins were precipitated with the addition of 0.3 mL of ice-cold methanol. The total mixture was centrifuged (14000 rpm for 15 min) to remove the proteins and the clear supernatant was analysed on reverse phase HPLC (RP-HPLC). The solvent system used was methanol:water:acetic acid in the ratio of 85:15:0.1 with a flow rate of 1 mL/min on a C18 column. The chromatograms were followed by monitoring the absorbance for HETE products at 235 nm. Individual peak areas in each chromatogram were taken for further comparison analysis. FIG. 2 shows the HPLC chromatogram of 12R-met-HETE and 8R-met-HETE generated after enzyme assay along with auto-oxidation substrate control.

### Screening of Compounds Against 12R-LOX in Isolated Enzyme Systems

Baicalein, the standard 12R-LOX inhibitor, was used as a positive control for all inhibition assays performed. The compounds were evaluated for their inhibitory action against human 12R-LOX enzyme at 100 μM concentration in vitro. Further, IC₅₀ of the compounds of the present invention were determined. The % inhibition at 100 μM concentration, and the IC₅₀ values of the compounds were summarized in Table 2 below.

TABLE 2

| | 12R-LOX inhibitory | |
|---|---|---|
| | activities in vitro | |
| Compound | % Inhibition | |
| No. | (at 100 μM) | IC₅₀ (μM) |
| 1 | 40 | 188 |
| 2 | 56 | |
| 3 | 51 | 97 |
| 7 | 21 ± 7 | |
| 13 | 21.02 ± 8.4 | |
| 14 | 6.95 ± 1.6 | |
| 15 | 6.33 ± 5.9 | |
| 16 | 38.33 ± 3.8 | |
| 17 | 30 ± 5 | |
| 18 | 53.45 ± 4.2 | 98.14 |

1: A compound of formula (I): 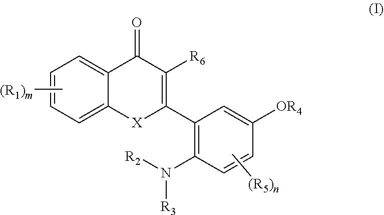
or a pharmaceutically acceptable salt or a stereoisomer thereof; wherein,
each of R₁, R₅ and R₆, at each occurrence, is independently selected from hydrogen, halogen, optionally substituted amine, optionally substituted alkyl, optionally substituted alkenyl, optionally substituted alkynyl, optionally substituted alkoxyalkyl, optionally substituted alkoxy, optionally substituted cycloalkyl, optionally substituted acyl, optionally substituted alkylsulfonyl, optionally substituted arylsulfonyl, optionally substituted heteroarylsulfonyl, optionally substituted aryl, —OR, and —COOR;
R₂ and R₃ are independently selected from hydrogen, optionally substituted alkyl, optionally substituted alkenyl, optionally substituted alkynyl, optionally substituted cycloalkyl, optionally substituted alkylsulfonyl, optionally substituted arylsulfonyl, optionally substituted heteroarylsulfonyl, optionally substituted aryl, optionally substituted arylalkyl, —CO-(optionally substituted alkyl), —CO-(optionally substituted aryl), optionally substituted heterocyclyl and optionally substituted heteroaryl, wherein the optional substituent, at each occurrence, is independently selected from one or more halogen, hydroxyl, alkyl, alkoxyalkyl, haloalkyl, alkoxy, nitro, cycloalkyl, alkylsulfonyl, arylsulfonyl, and —COOR; or
R₂ and R₃ together with the nitrogen to which they attached form an imine of formula, —N═C(R₂ₐ)(R_{2b}), wherein R₂ₐ and R_{2b} are independently selected from hydrogen, alkyl and aryl;
R₄ is selected from hydrogen, optionally substituted alkyl, optionally substituted alkenyl, optionally substituted alkynyl, optionally substituted alkoxyalkyl, optionally optionally substituted cycloalkyl, optionally substituted aryl, substituted alkylsulfonyl and optionally substituted arylsulfonyl;
R is hydrogen, optionally substituted alkyl or optionally substituted aryl;
X is oxygen, sulfur or NR₇; wherein R₇ is hydrogen, halogen, hydroxyl, optionally substituted alkyl, optionally substituted alkoxyalkyl, optionally substituted alkoxy, optionally substituted cycloalkyl, optionally substituted acyl, optionally substituted alkylsulfonyl, optionally substituted arylsulfonyl, optionally substituted heteroarylsulfonyl, optionally substituted aryl, optionally substituted heterocyclyl and optionally substituted heteroaryl;
m is 1, 2, 3, or 4; and
n is 1, 2 or 3.

2: The compound as claimed in claim 1 is a compound of formula (IA): 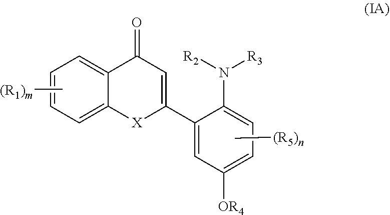
or a pharmaceutically acceptable salt or a stereoisomer thereof, wherein R₁, R₂, R₃, R₄, R₅, X, m and n are same as defined in claim 1.

3: The compound as claimed in claim 1 is a compound of formula (IB): 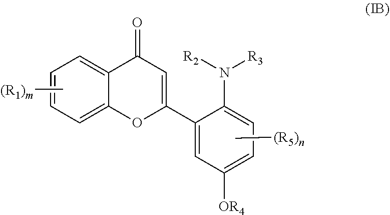
or a pharmaceutically acceptable salt or a stereoisomer thereof, wherein R₁, R₂, R₃, R₄, R₅, m and n are same as defined in claim 1.

4: The compound as claimed in claim 1 is a compound of formula (ID) or (IE): 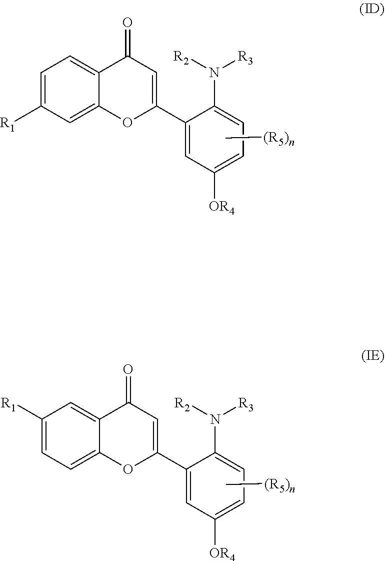
or a pharmaceutically acceptable salt or a stereoisomer thereof, wherein R₁, R₂, R₃, R₄, R₅ and n are same as defined in claim 1

5: The compound as claimed in claim 1,
wherein R₁ is independently selected from hydrogen, halogen or hydroxyl.

6: The compound as claimed in claim 1,
wherein R₅ is hydrogen or alkoxy.

7: The compound as claimed in claim 1 is a compound of formula (IC): 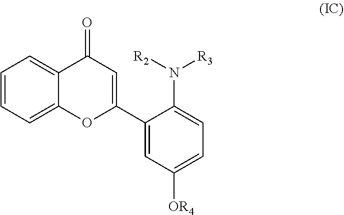
or a pharmaceutically acceptable salt or a stereoisomer thereof, wherein R₂, R₃, and R₄ are same as defined in claim 1.

8: The compound as claimed in claim 1 or a pharmaceutically acceptable salt or a stereoisomer thereof, wherein R₂ and R₃ are independently selected from hydrogen, optionally substituted alkylsulfonyl, or optionally substituted arylsulfonyl and CO-aryl, wherein the optional substituent is selected from one or more of nitro, alkyl and haloalkyl.

9: The compound as claimed in claim 8 or a pharmaceutically acceptable salt or a stereoisomer thereof, wherein aryl is phenyl.

10: The compound as claimed in claim 1 or a pharmaceutically acceptable salt or a stereoisomer thereof, wherein R₄ is selected from hydrogen, alkyl, alkynyl and alkylsulfonyl.

11: A compound selected from:

| Compound | |
|---|---|
| No. | Structure |
| 1 | 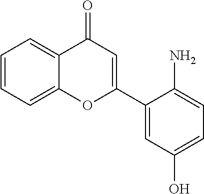 [SMILES: Nc1ccc(O)cc1-c1cc(=O)c2ccccc2o1] |
| 2 | 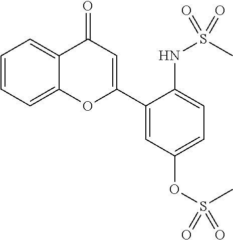 [SMILES: CS(=O)(=O)Nc1ccc(OS(C)(=O)=O)cc1-c1cc(=O)c2ccccc2o1] |
| 3 | 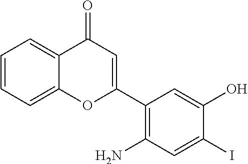 [SMILES: Nc1cc(I)c(O)cc1-c1cc(=O)c2ccccc2o1] |
| 4 | 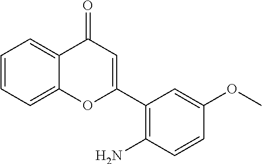 [SMILES: COc1ccc(N)c(-c2cc(=O)c3ccccc3o2)c1] |
| 5 | 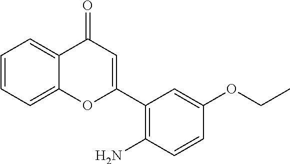 [SMILES: CCOc1ccc(N)c(-c2cc(=O)c3ccccc3o2)c1] |
| 6 | 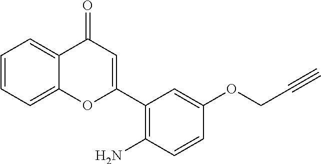 [SMILES: C#CCOc1ccc(N)c(-c2cc(=O)c3ccccc3o2)c1] |
| 7 | 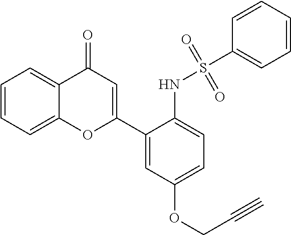 [SMILES: C#CCOc1ccc(NS(=O)(=O)c2ccccc2)c(-c2cc(=O)c3ccccc3o2)c1] |
| 8 | 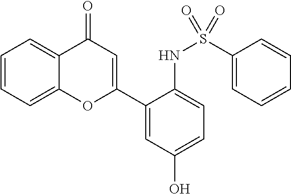 [SMILES: O=c1cc(-c2cc(O)ccc2NS(=O)(=O)c2ccccc2)oc2ccccc12] |
| 9 | 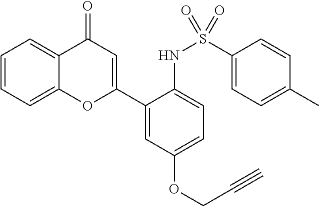 [SMILES: C#CCOc1ccc(NS(=O)(=O)c2ccc(C)cc2)c(-c2cc(=O)c3ccccc3o2)c1] |
| 10 | 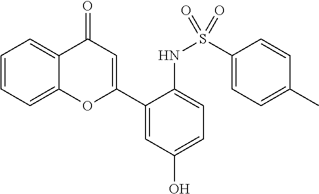 [SMILES: Cc1ccc(S(=O)(=O)Nc2ccc(O)cc2-c2cc(=O)c3ccccc3o2)cc1] |
| 11 | 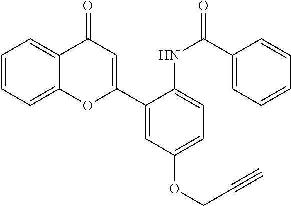 [SMILES: C#CCOc1ccc(NC(=O)c2ccccc2)c(-c2cc(=O)c3ccccc3o2)c1] |
| 12 | 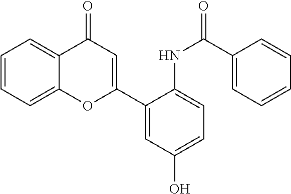 [SMILES: O=C(Nc1ccc(O)cc1-c1cc(=O)c2ccccc2o1)c1ccccc1] |
| 13 | 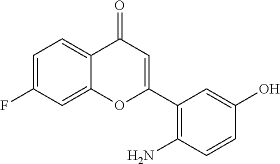 [SMILES: Nc1ccc(O)cc1-c1cc(=O)c2ccc(F)cc2o1] |
| 14 | 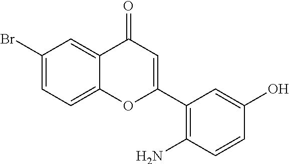 [SMILES: Nc1ccc(O)cc1-c1cc(=O)c2cc(Br)ccc2o1] |
| 15 | 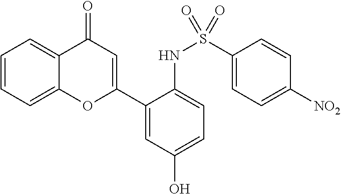 [SMILES: O=c1cc(-c2cc(O)ccc2NS(=O)(=O)c2ccc([N+](=O)[O-])cc2)oc2ccccc12] |
| 16 | 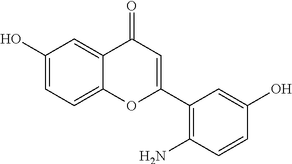 [SMILES: Nc1ccc(O)cc1-c1cc(=O)c2cc(O)ccc2o1] |
| 17 | 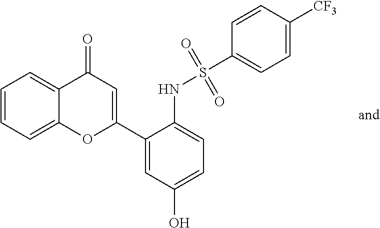 [SMILES: O=c1cc(-c2cc(O)ccc2NS(=O)(=O)c2ccc(C(F)(F)F)cc2)oc2ccccc12 ; "and"] |
| 18 | 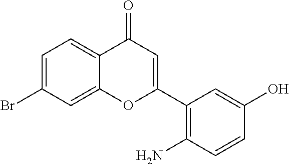 [SMILES: Nc1ccc(O)cc1-c1cc(=O)c2ccc(Br)cc2o1] |

or a pharmaceutically acceptable salt or a stereoisomer thereof

12: A pharmaceutical composition comprising a therapeutically effective amount of one or more compounds as claimed in claim 1 or a pharmaceutically acceptable salt or a stereoisomer thereof and one or more pharmaceutically acceptable carriers.

13. (canceled)

14: A method for treating or preventing a disease and/or disorder mediated by aberrant or undesired expression of one or more LOXs, comprising administering a therapeutically or prophylactically effective amount of a compound as claimed in claim 1 to a subject in need thereof.

15: The method as claimed in claim 14, wherein LOX mediated disease and/or disorder is selected from a group comprising Alzheimer's disease, cancer, cardiovascular disease, diabetes (type 1 and/or type 2), diabetic kidney disease, diabetic nerve disease, heparin induced thrombocytopenia, non-alcoholic steatohepatitis, platelet hemostasis, skin diseases, thrombosis, asthma, arthritis, ulcerative colitis, allergic diseases, auto-immune diseases, Parkinson's disease, atherosclerosis, hypertension, Schizophrenia and sepsis.

16: The method as claimed in claim 14, wherein LOX is 12R-LOX.

17: The method as claimed in claim 15, wherein skin disease is selected from a group comprising ichthyosis, psoriasis, skin cancers, autosomal recessive congenital ichthyosis (ARCI) and systematic sclerosis.
